(19) United States
(12) Patent Application Publication
(10) Pub. No.: US 2023/0235036 A1
(43) Pub. Date: Jul. 27, 2023

(54) ACVR1 (ALK2) RECEPTOR INHIBITION TO TREAT NEUROLOGICAL DISEASES

(71) Applicants: The J. David Gladstone Institutes, a testamentary trust established under the Will of J. David Glads, San Francisco, CA (US); The Regents of the University of California, Oakland, CA (US)

(72) Inventors: Katerina Akassoglou, San Francisco, CA (US); Mark Petersen, North Vancouver AB (CA)

(21) Appl. No.: 18/008,324

(22) PCT Filed: Jun. 4, 2021
(86) PCT No.: PCT/US2021/036023

§ 371 Date: Dec. 5, 2022

Related U.S. Application Data
(60) Provisional application No. 63/035,538, filed on Jun. 5, 2020.

(51) Int. Cl. C07K 16/22; A61K 45/06

(52) U.S. Cl. CPC C07K 16/22; A61K 45/06

(57) ABSTRACT

Compositions and methods to treat or prevent neurodegeneration in a mammal comprising administering to the mammal in need thereof an effective amount of an inhibitor of ACVR1 (Alk2).

### CLAIM OF PRIORITY

This application claims the benefit of priority of U.S. Provisional Patent Application No. 63/035,538, filed on Jun. 5, 2020, the benefit of priority of which is claimed hereby, and which is incorporated by reference herein in its entirety.

### GOVERNMENT FUNDING

This invention was made with Government support under W81XWH-17-1-0211 awarded by the ARMY/MRMC and under R35 NS097976 awarded by the National Institutes of Health. The Government has certain rights in the invention.

### BACKGROUND OF THE INVENTION

In the following discussion certain articles and methods will be described for background and introductory purposes. Nothing contained herein is to be construed as an “admission” of prior art. Applicant expressly reserves the right to demonstrate, where appropriate, that the articles and methods referenced herein do not constitute prior art under the applicable statutory provisions.

Neurodegeneration is the progressive loss of structure and/or function of neurons, which may lead to the death of the affected neurons. Neurodegenerative diseases include Alzheimer's disease, Parkinson's disease, amyotrophic lateral sclerosis, Huntington's disease and multiple sclerosis. Although these diseases have different etiologies and symptoms, they all result in progressive degeneration and/or death of neuron cells. Despite their differences, these diseases also display similarities that can relate these diseases on a cellular or molecular level. Myelin abnormalities and inhibition of remyelination are present in many of these diseases. Such similarities offer therapeutic advances using modalities common to each of these diseases.

Clinical management of neurodegenerative remains a significant challenge in medicine, however, as they do not address the cellular or molecular basis of the disease. Although some degree of axonal remyelination by oligodendrocytes takes place early during the course of MS, the ability to repair the CNS eventually fails, leading to irreversible tissue injury and an increase in disease-related disabilities. Currently approved therapies for CNS demyelinating diseases, such as multiple sclerosis (MS), are primarily immunomodulatory, and typically do not have direct effects on CNS repair. In addition to MS, myelin abnormalities are present in Alzheimer's disease. Similarly, drugs for other neurodegenerative diseases such as Alzheimer's disease and Parkinson's disease do not address the neuronal death and loss of function, but rather ameliorate associated symptoms.

Thus, there is a need for additional therapies that promote neurorepair, prevent and/or ameliorate neurodegeneration.

### SUMMARY OF THE INVENTION

Provided herein are method and compositions for treating and preventing neurodegeneration and promoting neurorepair.

One embodiment provides a method to treat or prevent neurodegeneration in a mammal comprising administering to the mammal in need thereof an effective amount of an inhibitor of at least one bone morphogenetic protein (BMP) receptor.

Another embodiment provides a method to treat or prevent neurodegeneration in a mammal comprising administering to the mammal in need thereof an effective amount of an inhibitor of ACVR1 (Alk2) or an agent that modulate the ligand for ACVR1 (activin).

One embodiment provides a method to promote remyelination in neurological diseases or disorders in a mammal, comprising administering to the mammal in need thereof an effective amount of an inhibitor of ACVR1 (Alk2) or an agent that modulate the ligand for ACVR1 (activin).

Another embodiment provides a method to prevent or ameliorate demyelination in a mammal comprising administering to the mammal in need thereof an effective amount of an inhibitor of ACVR1 (Alk2) or an agent that modulate the ligand for ACVR1 (activin).

One embodiment provides a method to enhance myelination and/or re-myelination in a mammalian subject, such as a human subject, by administering to the mammal in need thereof an effective amount of an inhibitor of ACVR1 (Alk2) or an agent that modulate the ligand for ACVR1 (activin).

One embodiment provides a method to decrease differentiation of progenitors to astrocytes in a mammalian subject, such as a human subject, by administering to the mammal in need thereof an effective amount of an inhibitor of ACVR1 (Alk2) or an agent that modulate the ligand for ACVR1 (activin).

In one embodiment, the inhibitor is of ACVR1 (Alk2) is LDN-212854, dorsomorphin, DMH1, saracatinib, BCX9250, KER-047, INCB000928, BLU-782, momelotinib, LDN-193189, K02288, LDN-214117, LDN-213844, M4K2009, M4K2149 or derivatives or variants thereof.

In one embodiment, the mammal is human.

In another embodiment, the mammal has been diagnosed with a disease, disorder, or injury involving demyelination, dysmyelination, or neurodegeneration. In one embodiment, said disease, disorder, or injury is selected from the group consisting of multiple sclerosis (MS), progressive multifocal leukoencephalopathy (PML), encephalomyelitis (EPL), central pontine myelolysis (CPM), adrenoleukodystrophy, Alexander's disease, Pelizaeus Merzbacher disease (PMZ), Wallerian Degeneration, optic neuritis, transverse myelitis, amyotrophic lateral sclerosis (ALS), Huntington's disease, Alzheimer's disease, Parkinson's disease, spinal cord injury, traumatic brain injury, neonatal brain injury, post radiation injury, neurologic complications of chemotherapy, stroke, acute ischemic optic neuropathy, vitamin E deficiency, isolated vitamin E deficiency syndrome, AR, Bassen-Kornzweig syndrome, Marchiafava-Bignami syndrome, metachromatic leukodystrophy, trigeminal neuralgia, acute disseminated encephalitis, Guillain-Barre syndrome, Marie-Charcot-Tooth disease and Bell's palsy.

In one embodiment, an additional agent is administered in the treatment of Alzheimer's disease, wherein said additional agent is an acetylcholinesterase inhibitor (e.g., donepezil, galantamine, and rivastigmine) and/or NMDA receptor antagonist (e.g., memantine).

In another embodiment, an additional agent is administered in the treatment of ALS, wherein said additional agent is Riluzole (Rilutek), minocycline, insulin-like growth factor 1 (IGF-1), and/or methylcobalamin.

In another embodiment, an additional agent is administered in the treatment of Parkinson's disease, wherein said additional agent is a L-dopa, dopamine agonist (e.g., bromocriptine, pergolide, pramipexole, ropinirole, cabergoline, apomorphine, and lisuride), dopa decarboxylase inhibitor (e.g., levodopa, benserazide, and carbidopa), and/or MAO-B inhibitor (e.g., selegiline and rasagiline).

In one embodiment, an additional agent is administered in the treatment of demyelinating diseases, wherein said additional agent is an interferon beta la inhibitor, interferon beta lb inhibitor, glatiramer acetate, daclizumab, clemastine, teriflunomide, fingolimod, dimethyl fumarate; alemtuzumab, mitoxantrone, and/or natalizumab.

One embodiment further comprises administering an additional promyelinating agent/drug. In one embodiment, the promyelinating agent/drug is a promyelinating benztropine, clemastine, quetiapine, miconazole, clobetasol, (±)U-50488, and XAV-939.

In one embodiment, the agent that modulates the ligand for ACVR1 (activin) is an antibody, such as REGN2477 (Regeneron; ifopa.org/regn2477).

### BRIEF DESCRIPTION OF THE FIGURES

A better understanding of the features and advantages of the present invention will be obtained by reference to the following detailed description that sets forth illustrative embodiments, in which the principles of the invention are utilized, and the accompanying drawings of which:

FIGS. 1A-G. NG2 cells cluster perivascularly at sites of fibrinogen deposition and limited remyelination in chronic neuroinflammation. A, In vivo 2P maximum intensity projection images of microglia (green), NG2 cells (red) and the vasculature (blue, 70 kDa Oregon Green Dextran) in NG2-CreER™:Rosa^{tdTomato/+}:Cx3cr1^{GFP/+} age-matched healthy control mice, at the peak of clinical signs (peak EAE, mean score 3) and at chronic EAE (mean clinical score 2.1). Images shown are from mice on days 17 (peak) and 35 (chronic) after the induction of EAE. An NG2^{tdTomato+} pericyte in the control condition is depicted with a white arrow. Scale bar, 50 μm. Quantification of NG2 cell and microglial clusters in control (n=4 mice), peak (n=5 mice) and chronic (n=6 mice) EAE. Values are mean±s.e.m., *p<0.05, n.s. not significant, (two-way ANOVA with Bonferroni's multiple comparisons test). B, Microscopy of spinal cord sections from unimmunized healthy mice (control) and MOG₃₅₋₅₅-EAE mice at peak and chronic stages of disease immunostained for fibrinogen (green). Nuclei are stained with 4′,6-diamidino-2-phenylindole (DAPI, blue). Scale bar, 100 μm. Quantification of dextran leakage in spinal cord of unimmunized, healthy mice (control) (n=4 mice) and MOG35-55-EAE mice at peak (n=5 mice) and chronic (n=6 mice) stages of disease. Values are mean±sem., *p<0.05 (one-way ANOVA with Tukey's multiple comparisons test). Quantification of fibrinogen immunoreactivity in spinal cord of unimmunized healthy mice (control) and MOG₃₅₋₅₅-EAE mice at peak and chronic stages of disease (n=3 mice per group). Values are mean±sem., **p<0.01, ***p<0.001 (one-way ANOVA with Tukey's multiple comparisons test). C, Microscopy of ventral spinal cord sections of NG2-CreER™:Rosa^{tdTomato/+}:Cx3cr1^{GFP/+} mice at chronic EAE immunostained for fibrinogen (green). Scale bar, 50 μm. Quantification of fibrinogen immunopositivity in areas of NG2 clusters and areas without clusters (n=5 mice). Values are mean±s.e.m., **p<0.01 (two-tailed Mann-Whitney test). D, In vivo 2P maximum intensity projection images of myelin (green) in NG2-CreER™:Rosa^{tdTomato/+}:Cx3cr1^{GFP/+} mice at chronic EAE in areas of NG2 clusters and areas without clusters. Boxed areas are shown in top right insets to only depict myelin labeling. Scale bar, 20 μm. Quantification of myelin circularity at chronic EAE in areas of NG2 clusters and areas without clusters (n=5 mice). Values are mean±s.e.m., **p<0.01 (two-tailed Mann-Whitney test). A value of 1.0 indicates a perfect circle (as seen in degenerating myelin in longitudinal sections); as the value approaches 0.0, it indicates an increasingly noncircular, linear shape (longitudinal section of normal myelinated fiber). E, ROI tracking workflow for the co-registration of 2P and SBEM volumes. F-G, Representative co-related SBEM images from n=3 ROIs from 2 different mice. Fi, CNS parenchyma in areas of NG2 clusters shows an inflamed spinal cord vessel with activated endothelial cells (green asterisk), attachment of a leukocyte to the endothelium (black arrowhead) and perivascular lesions with dominant demyelination (red boxed area) and sparse remyelination (blue boxed area). Scale bar, 20 μm. Fii, red boxed area is shown at higher magnification. Red arrows depict demyelinated axons. Scale bar, 10 μm. Fiii, blue boxed area is shown at higher magnification. Blue arrows depict remyelinated axons. Scale bar, 10 μm. Fiv, Correlated SBEM within the CNS parenchyma in an area without NG2 clusters. Black arrows depict normal myelinated axons. Scale bar, 10 μm. Gi, Representative SBEM from another ROI in an area of NG2 cluster shows a vein with perivascular demyelination, gliosis (red dotted area) and some limited remyelination (blue boxed area). The area of gliosis contains an infiltrating macrophage (M) and an astrocyte (A). Distal areas have normal myelinated axons depicted with black arrows. Scale Bar, 10 μm. Gii, blue boxed area is shown at higher magnification. Blue arrows depict remyelinated axons. Black arrowheads depict NG2 cells. Scale Bar, 5 μm.

FIGS. 2A-F. RNA-seq analysis of NG2 cells in EAE reveals suppression of anticoagulation pathways. Data are from n=3 mice per group (A-D). A, Volcano plot of DEGs from RNA-seq analysis of NG2 lineage cells from MOG₃₅₋₅₅-EAE or healthy mice. Circles depict genes significantly downregulated (blue; log 2 fold change <−1; FDR<0.05) or upregulated (red; log 2 fold change >1; FDR<0.05) in EAE compared to healthy mice. B, Heat map of data from A. Genes were clustered by HOPACH unsupervised clustering analysis (Clusters 1-9). Expression values were log normalized, row centered and depicted as z-score. Significant GO terms and example genes are shown for each cluster. FDR<0.05; Benjamini-Hochberg correction. C, Visualization of co-expression GO term networks downregulated (blue nodes) or upregulated (red nodes) in NG2 cells from EAE compared to healthy mice. Gene set size and co-expression overlap (key) was determined by GSEA, p<0.05. D, Enrichment plot for the gene sets “Negative regulation of coagulation” and “Regulation of cell junction assembly” determined by GSEA of RNA-seq data of NG2 cells from EAE or healthy mice. X-axis depicts gene rank in dataset. NES, normalized enrichment score. E-F, Representative histograms of surface labeled TFPI and quantification of TFPI+ cells in PDGFRα+ OPC (E) or PDGFRβ+ pericyte (F) populations from healthy and EAE mice. Data are from n=5 per group (mean±s.e.m.) **p<0.01, n.s. not significant (two-tailed Mann-Whitney test).

FIGS. 3A-G. Promyelinating compounds do not overcome fibrinogen extrinsic inhibition of OPC differentiation. A, Workflow for medium throughput, OPC-X screen of promyelinating drugs in the presence of fibrinogen. B-C, Immunofluorescence for MBP (green) and GFAP (red) in primary rat OPCs treated with fibrinogen and myelin-promoting drugs or vehicle control (dimethylsulfoxide, DMSO) as indicated. Nuclei are stained with Hoechst dye (blue). Representative images from n=3 independent experiments. Scale bar, 100 μm. D-E, Quantification of percentage of total cells MBP+ or GFAP+ from automated image acquisition and quantification. Data are mean±s.e.m. from n=3 independent experiments. ****p<0.0001 (one-way ANOVA with Dunnett's multiple comparisons test). F, Phospho-SMAD1/5 (P-SMAD1/5) and ID2 protein levels in control or fibrinogen-treated primary rat OPCs in the presence of DMH1 or clemastine. Values are mean of n=3 independent experiments. G, Immunofluorescence for MBP (green) and GFAP (red) in primary rat OPCs treated with fibrinogen and LDN-212854 (0.18 μM) or vehicle control (DMSO) for three days. Nuclei are stained with Hoechst dye (blue). Representative images from n=3 independent experiments. Scale bar, 100 μm. H, Quantification of percentage of total MBP+ or GFAP+ cells from automated image acquisition and quantification. Data are mean±s.e.m. from n=3 independent experiments. *p<0.05, **p<0.01, ***p<0.001, ****p<0.0001 (matched oneway ANOVA with Dunnett's multiple comparisons test).

FIGS. 4A-E. Therapeutic effects of type I BMP receptor inhibition in chronic neuroinflammation. A, Clinical scores for MOG₃₅₋₅₅-EAE mice treated with LDN-212854 or saline (key) for 14 days starting at peak disease. Data are from n=6 mice (EAE+LDN-212854) and n=5 mice (EAE+saline), mean±s.e.m., *p<0.05, (two-tailed permutation test). B, Microscopy of spinal cord sections from MOG₃₅₋₅₅-EAE mice treated with saline (left panel) or LDN-212854 (right panel) immunostained for MBP to visualize myelin (green) and fibrinogen (red). Dashed line demarcates demyelinated white matter. Scale bar, 50 μm. Data are from n=5 mice per group, mean±s.e.m., **p<0.01 (two-tailed Mann-Whitney test). C, Clinical scores for NOD-MOG₃₅₋₅₅ EAE mice treated with LDN-212854 or saline (key) for 30 days. Data are from n=8 mice (EAE+LDN-212854) and n=7 mice (EAE+saline), mean±s.e.m., *p<0.05, (Welch two-sample t-test comparing the group means of maximum scores, Saline=2.36, LDN-212854=1.75). D, Microscopy of spinal cord sections from NOD-MOG35-55 EAE mice treated with saline (left panel) or LDN-212854 (right panel) with darkfield microscopy used to visualize myelin (green) and immunostained for fibrinogen (red). Dashed line demarcates demyelinated white matter. Scale bar, 100 μm. Data are from n=6 mice per group, mean±s.e.m., *p<0.05 **p<0.01 (two-tailed Mann-Whitney test). E, In vivo 2P maximum intensity projection images of NG2 cells (red) and the vasculature (blue, 70 kDa Oregon Green Dextran) in NG2-CreER™:Rosa^{tdTomato/+} mice at chronic EAE treated with saline (left panel) and LDN-212854 (right panel). Scale bar, 50 μm. Data are from n=6 (EAE+LDN-212854) and n=5 (EAE+saline), mean±s.e.m, *p<0.05 (two-tailed unpaired t-test). F, In vivo 2P maximum intensity projection images of NG2 cells (red) and myelin (green, MitoTracker) in NG2-CreER™:Rosa^{tdTomato/+} mice at chronic EAE treated with saline (left panel) and LDN-212854 (right panel). Scale bar, 20 μm. Data are from n=5 (EAE+LDN-212854) and n=4 (EAE+saline), mean±s.e.m., *p<0.05 (two-tailed Mann-Whitney test). Myelin damage was quantified with myelin circularity where a value of 1.0 indicates a perfect circle; as the value approaches 0.0, it indicates an increasingly noncircular shape, linear shape. G, Microscopy of spinal cord sections from NG2-CreER™:Rosa^{tdTomato/+} MOG₃₅₋₅₅-EAE mice after 14 day treatment of saline (left panel) or LDN-212854 (right panel). NG2 cells (red) and immunostaining for ID2 (green). Nuclei are stained with 4′,6-diamidino-2-phenylindole (DAPI, blue). Scale bar, 25 μm. Data are from n=6 (EAE+LDN-212854) and n=5 (EAE+saline), mean±s.e.m., **p<0.01 (twotailed Mann-Whitney test). H, Fate mapping of tdTomato⁺ OPC-derived cells using microscopy of spinal cord sections from NG2-CreER™:Rosa^{tdTomato/+} MOG₃₅₋₅₅-EAE mice after 14 day treatment of LDN-212854 or saline. NG2^{tdTomato/+} cells (red) and immunostaining for the mature OL marker GST-pi (green, top panel) or the astrocyte marker GFAP (green, bottom panel). Scale bar, 50 μm (top panel) and 20 μm (bottom panel). Data are from n=6 (EAE+LDN-212854) and n=5 (EAE+saline), mean±s.e.m., **p<0.01 (two-tailed Mann-Whitney test).

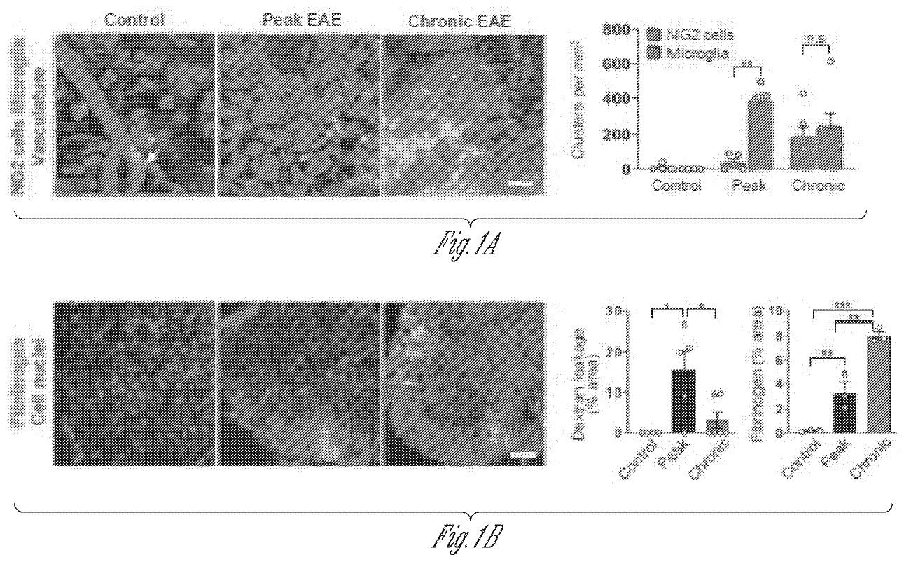

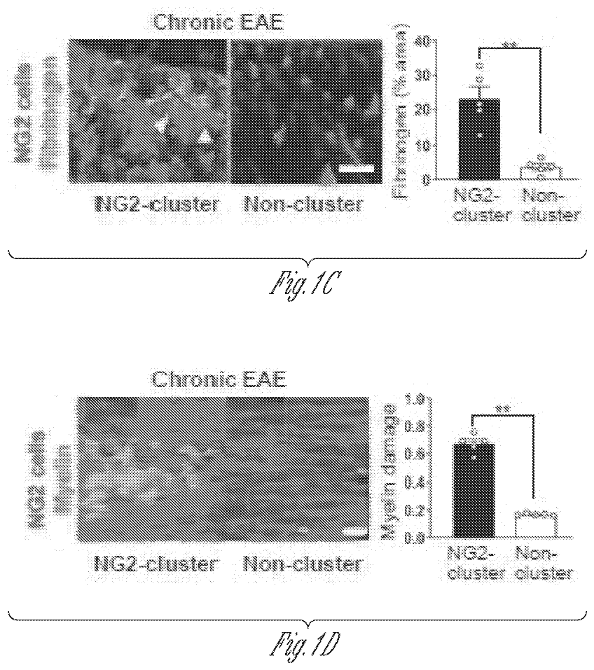

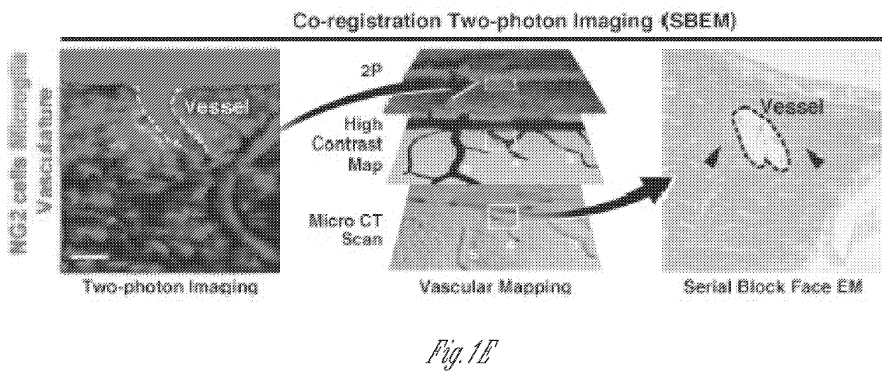

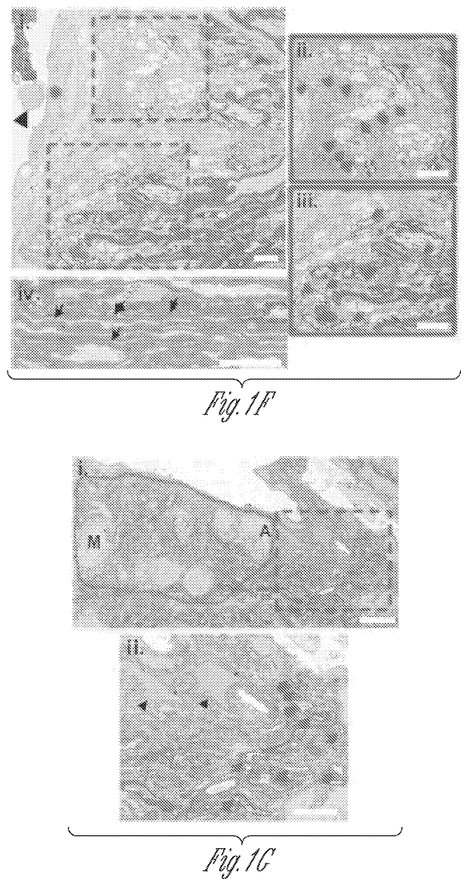

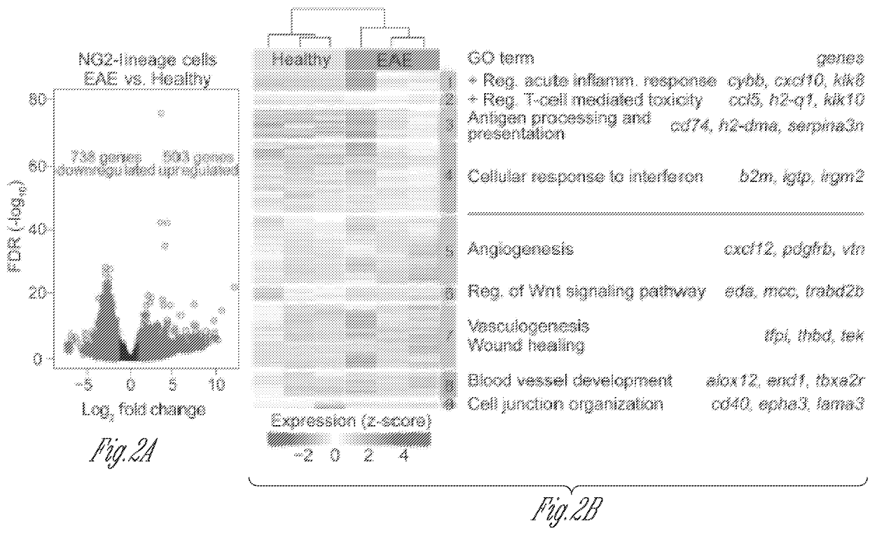

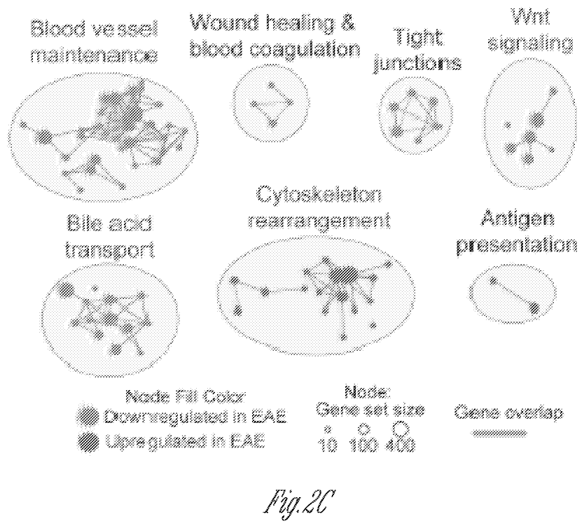

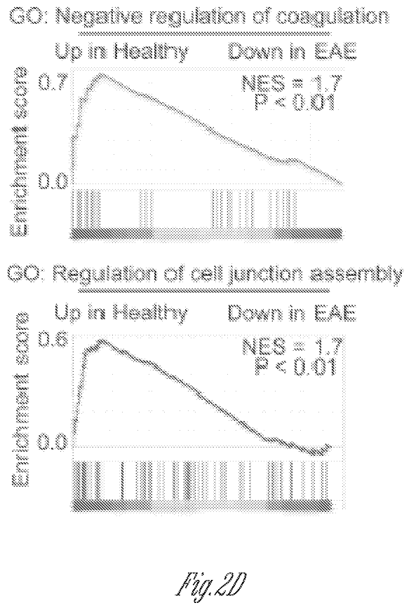

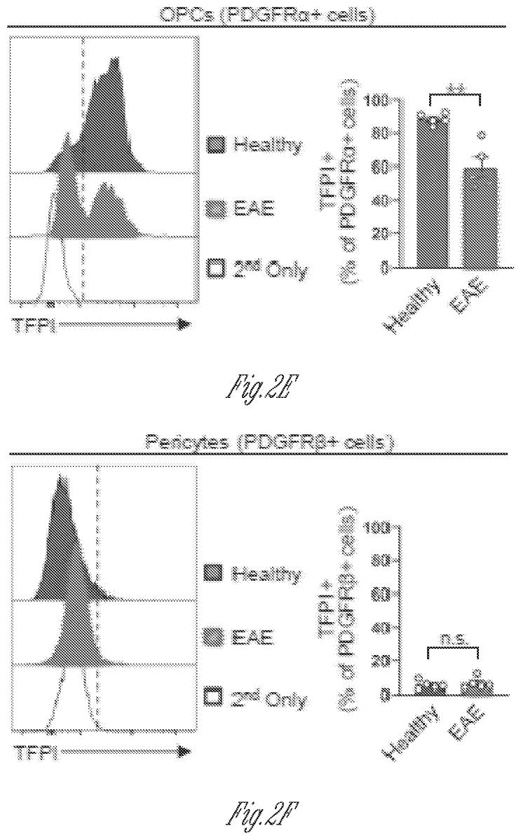

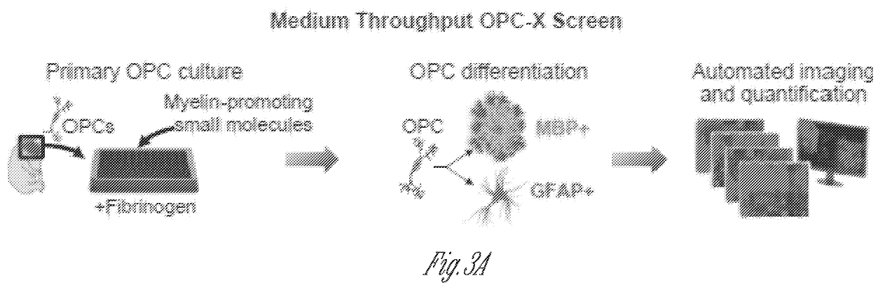

Supplementary FIG. 1. Workflow for in vivo 2P imaging and bulk RNA-seq analysis of NG2-lineage cells and microglia in NG2creER™:Rosa^{tdTomato/+}:Cx3cr1^{GFP/+} mice in MOG₃₅₋₅₅-EAE.

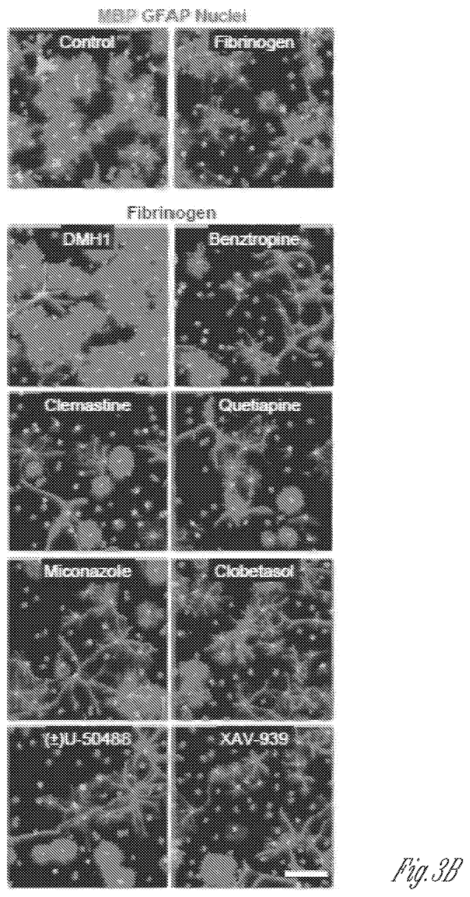

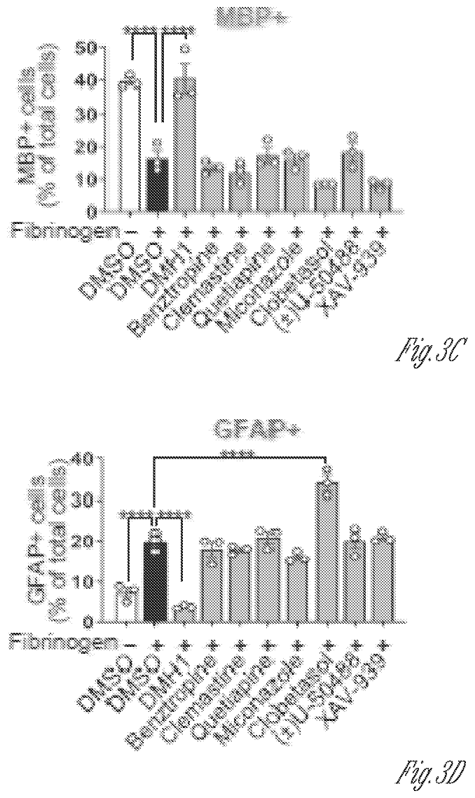

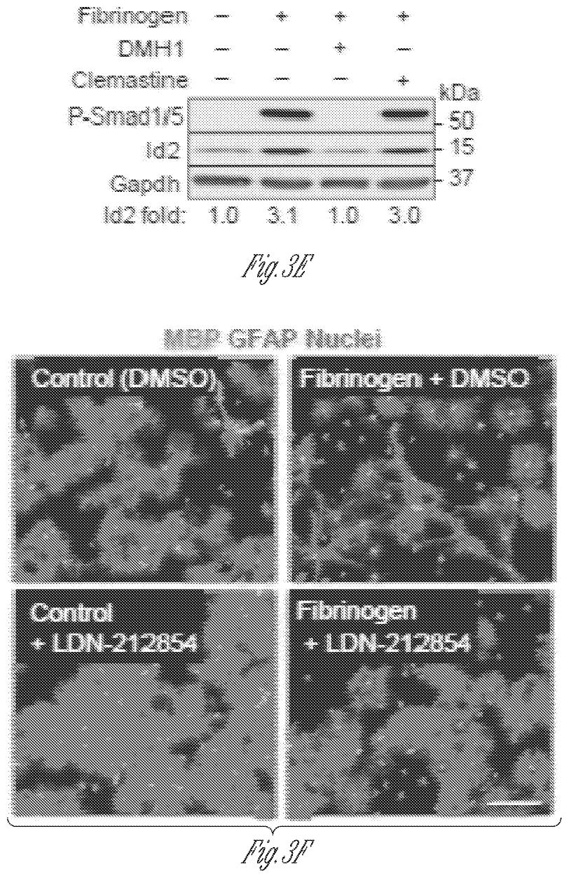

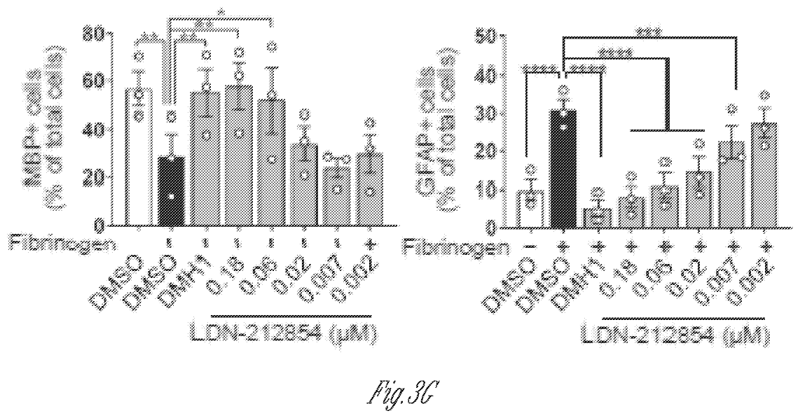

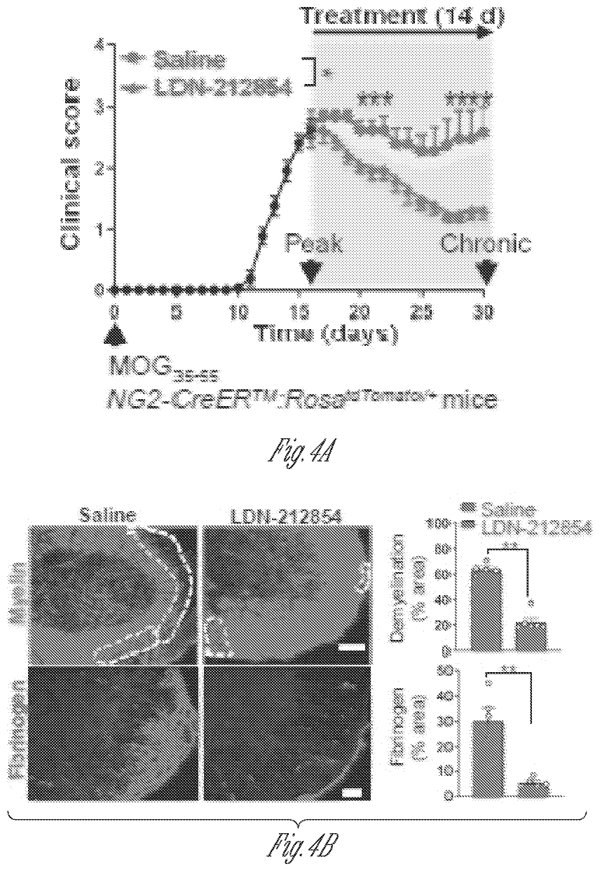

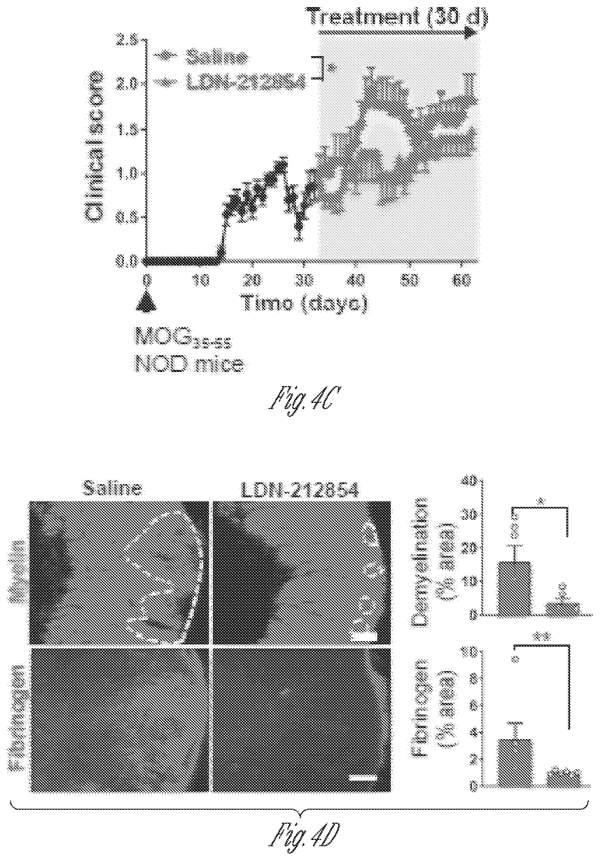

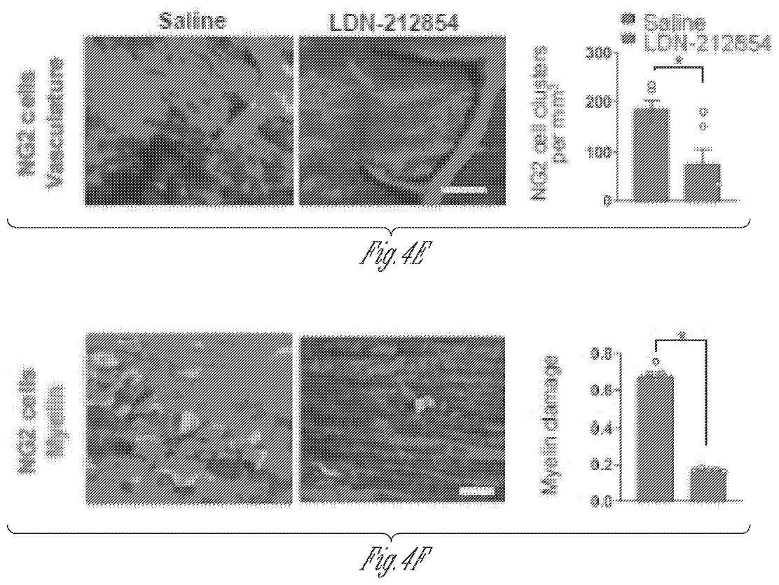

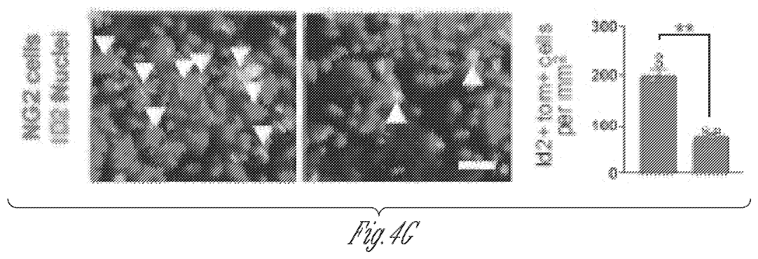

Supplementary FIGS. 2A-C. In vivo 2P imaging of NG2 cells and microglia at the neurovascular interface at different stages of EAE. In vivo 2P maximum intensity projection images of NG2 cells (red, top panel), microglia (green, bottom panel) and the vasculature (blue, 70 kDa Oregon Green Dextran) in NG2creER™:Rosa^{tdTomato/+}:Cx3cr1^{GFP/+} age-matched healthy control mice, at the peak of clinical signs (peak EAE, mean score 3) and at chronic EAE (mean clinical score 2.1). Scale bar, 100 μm. Quantification of co-localization of NG2 clusters and microglial clusters at peak (n=5 mice) and chronic (n=6 mice) EAE. Values are mean±s.e.m., **p<0.01 (two-tailed Mann-Whitney test). B, In vivo 2P maximum intensity projection images of NG2 cells (red) and the vasculature (blue, 70 kDa Oregon Green Dextran) in NG2creER™:Rosa^{tdTomato/+}:Cx3cr1^{GFP/+} age-matched healthy control mice, at the peak of clinical signs (peak EAE, mean score 3) and at chronic EAE (mean clinical score 2.1). Scale bar, 50 μm. Quantification of the distance of NG2 clusters from the nearest blood vessel at chronic EAE (data from 45 clusters in 6 mice). An NG2^{tdTomato/+} pericyte in the control condition is depicted with a white arrow. C, In vivo 2P maximum intensity projections of tdTomato⁺ (red) pericytes (left panel) and OL-lineage cell in relation to the vasculature (blue, 70 kDa Oregon Green Dextran) in the spinal cord parenchyma of NG2-CreER™:Rosa^{tdTomato/+}:Cx3cr1^{GFP/+} mice. Scale bar, 20 μm.

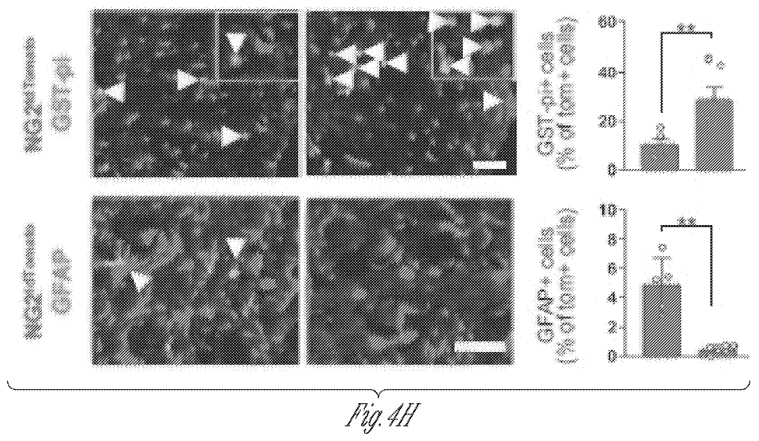

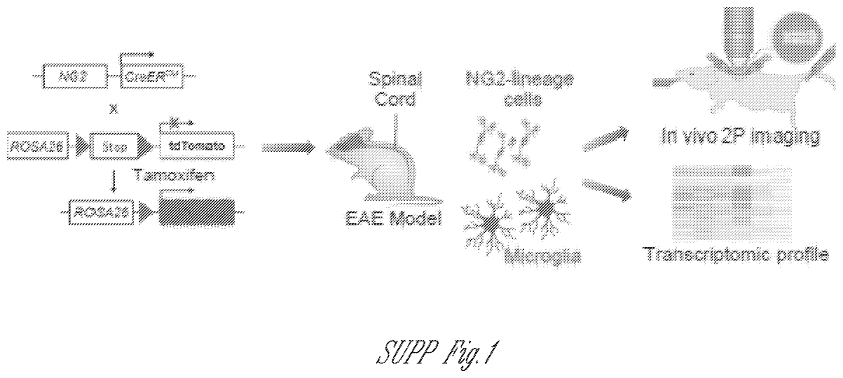

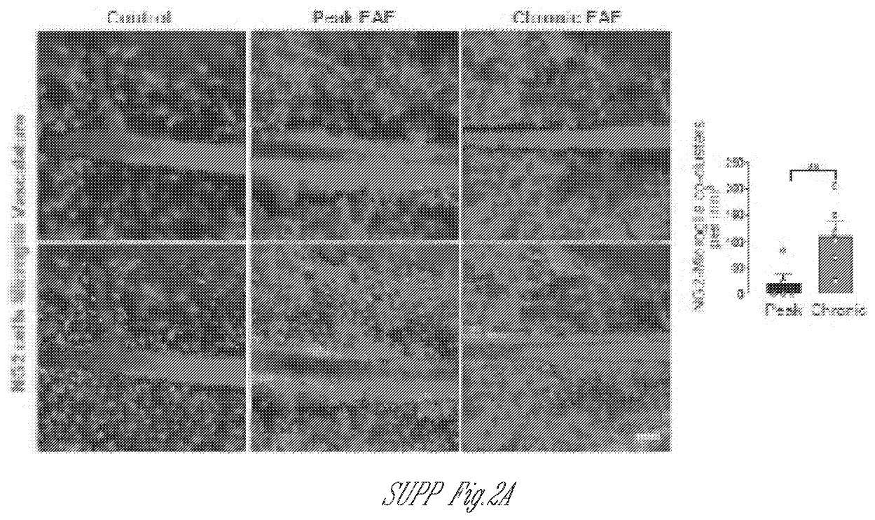

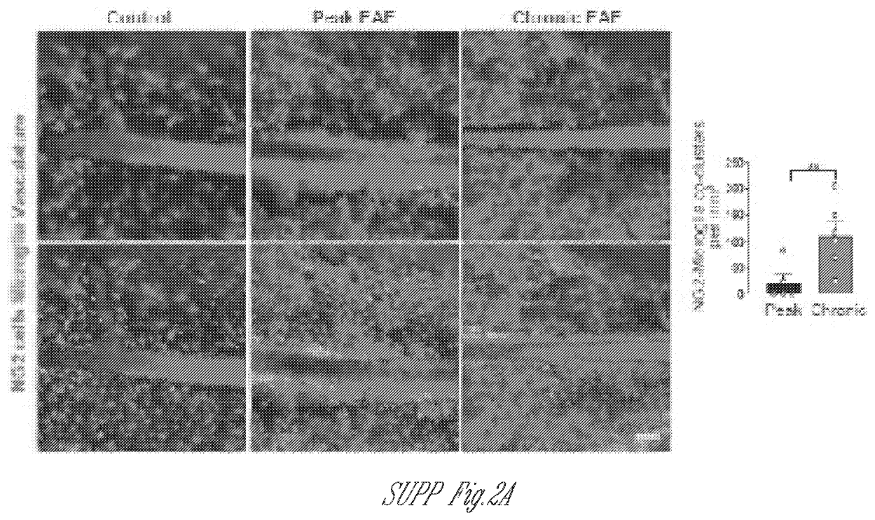

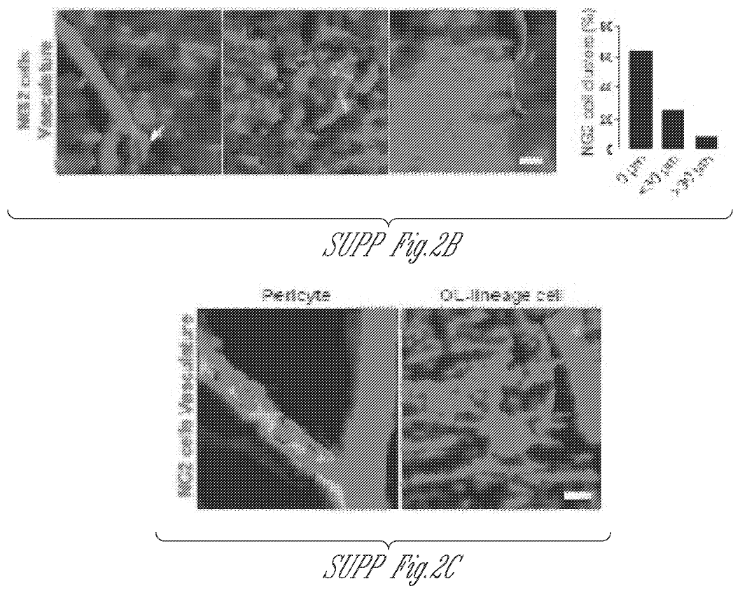

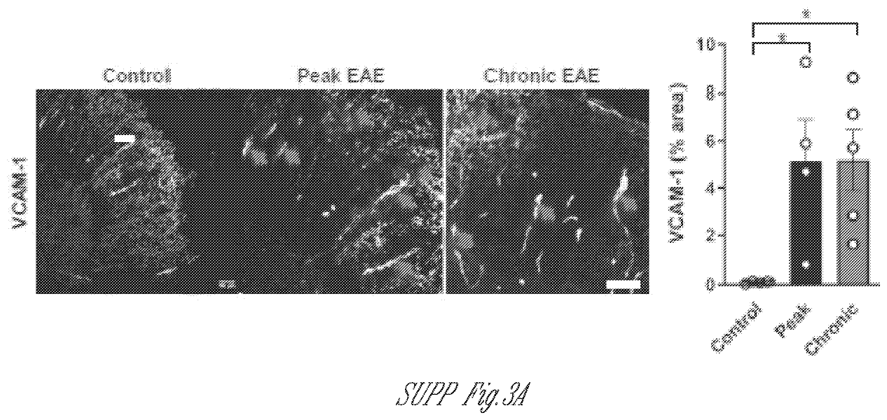

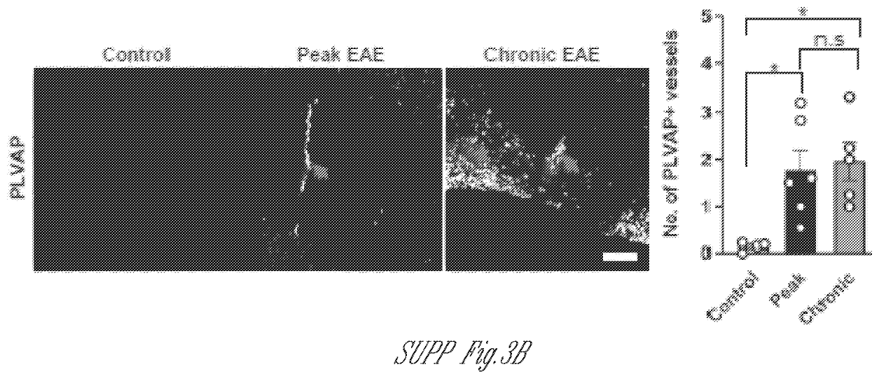

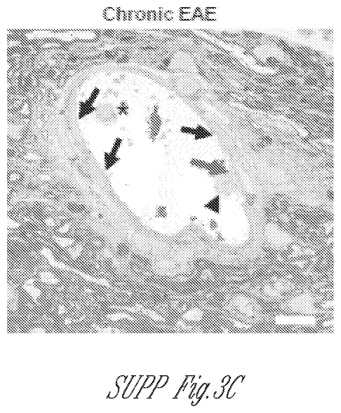

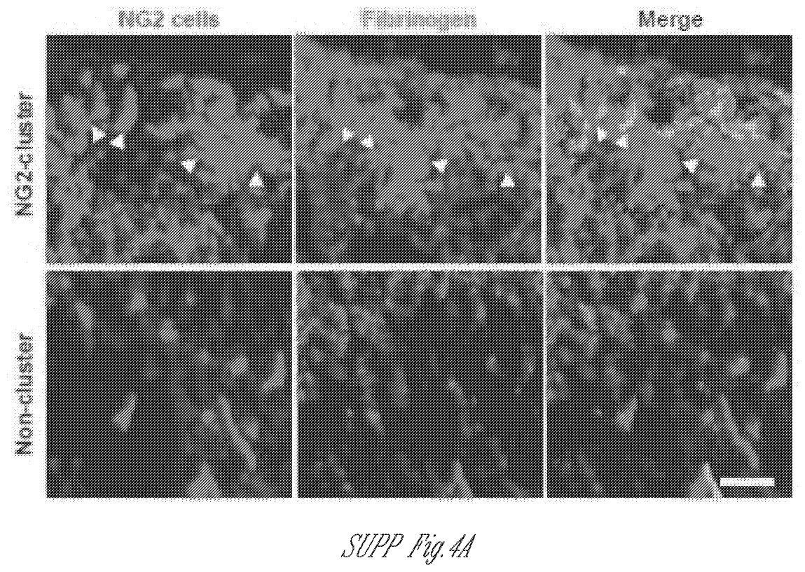

Supplementary FIGS. 3A-C. Endothelial activation at different stages of EAE. A, Microscopy of ventral spinal cord sections of NG2-CreER™:Rosa^{td/Tomato/+} mice in control, peak EAE and chronic EAE immunostained for VCAM-1. Red arrows depict vascular VCAM-1 expression; red asterisks depict diffuse VCAM-1 positivity. Quantification of VCAM-1 immunoreactivity in ventral spinal cord in control, peak EAE and chronic EAE. Scale bar, 50 μm. Values are mean±s.e.m., **p<0.05 (one-way ANOVA with Dunnett's multiple comparisons test). B, Microscopy of ventral spinal cord sections of NG2-CreER™:Rosa^{tdTomato/+} mice in control, peak EAE and chronic EAE immunostained for PLVAP. Red arrows depict vascular PLVAP expression; red asterisks depict diffuse PLVAP positivity. Scale bar, 50 μm. Quantification of PLVAP+ vessels in ventral spinal cord in control, peak EAE and chronic EAE. Values are mean±s.e.m., *p<0.05 (one-way ANOVA with Tukey's multiple comparisons test). C, CNS parenchyma in areas of NG2 clusters shows an inflamed spinal cord vessel with activated endothelial cells. Depicted here are activated endothelia (black arrows) which are thicker compared to the very thin endothelia in normal BBB vessels. These activated endothelia form small protrusions or processes (red arrow), which make contacts with leukocytes (black arrowhead) within the vessel.

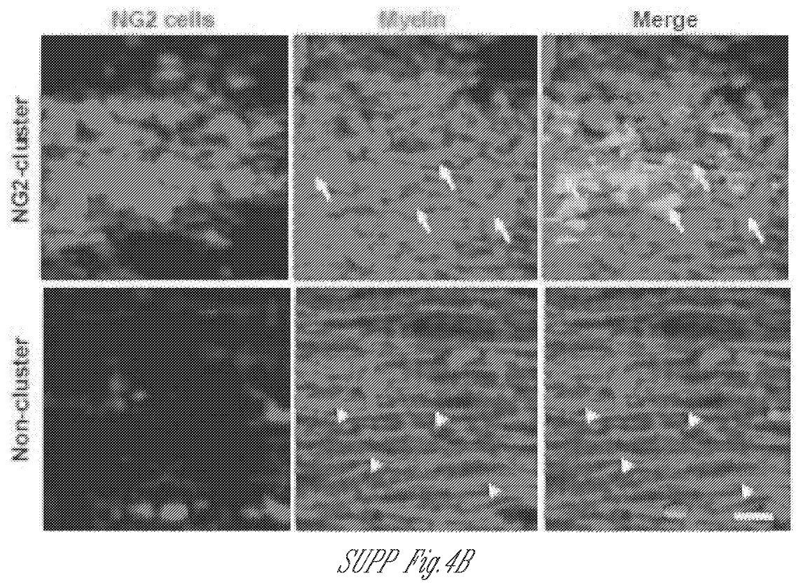

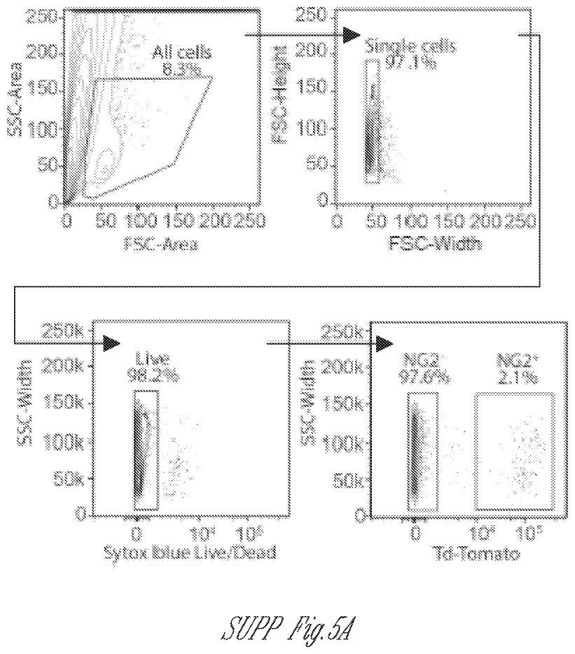

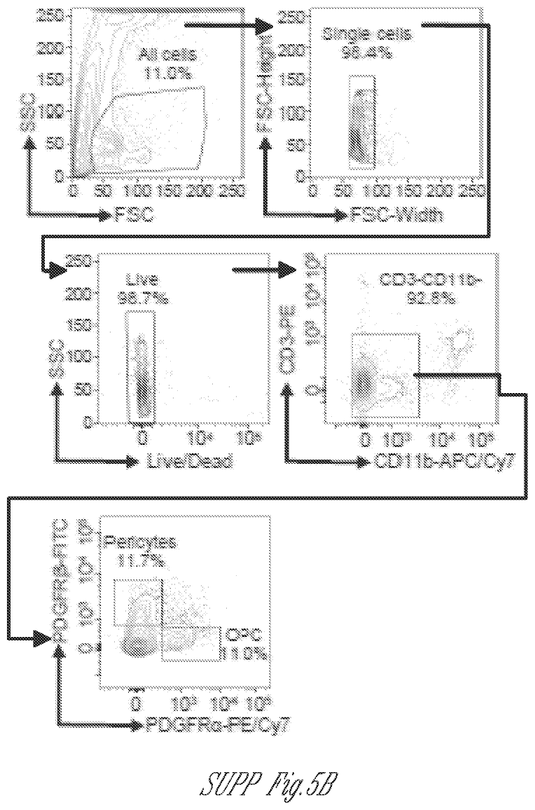

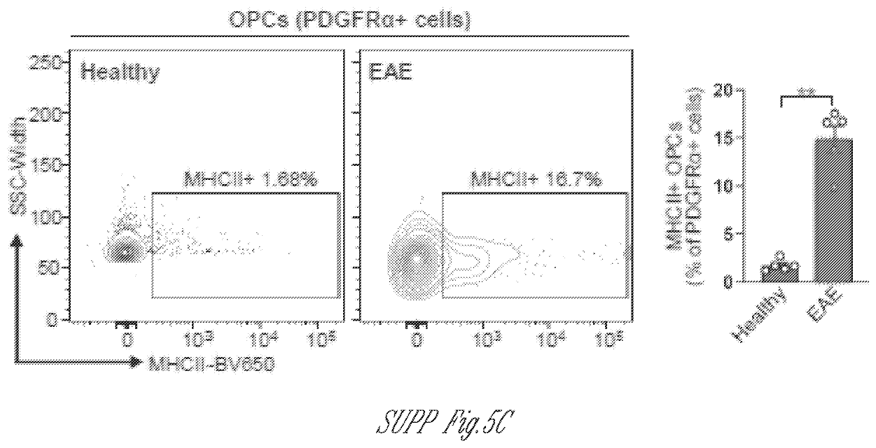

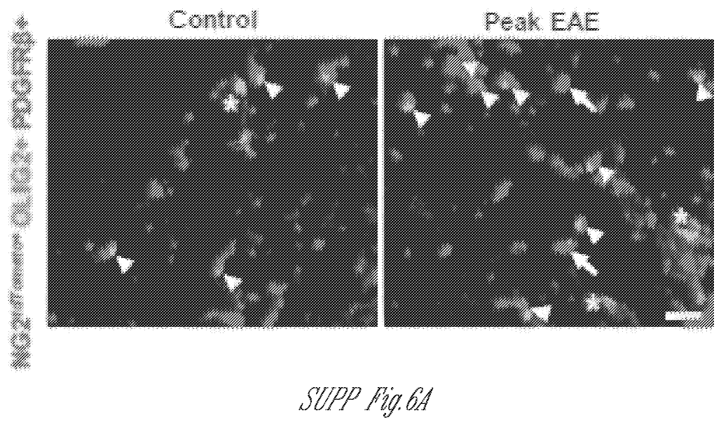

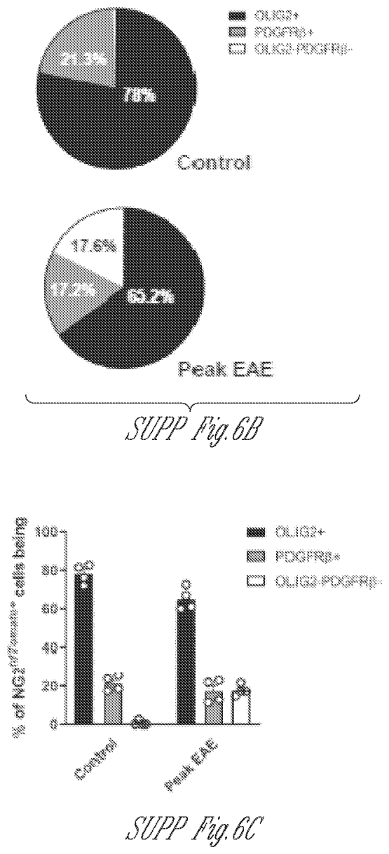

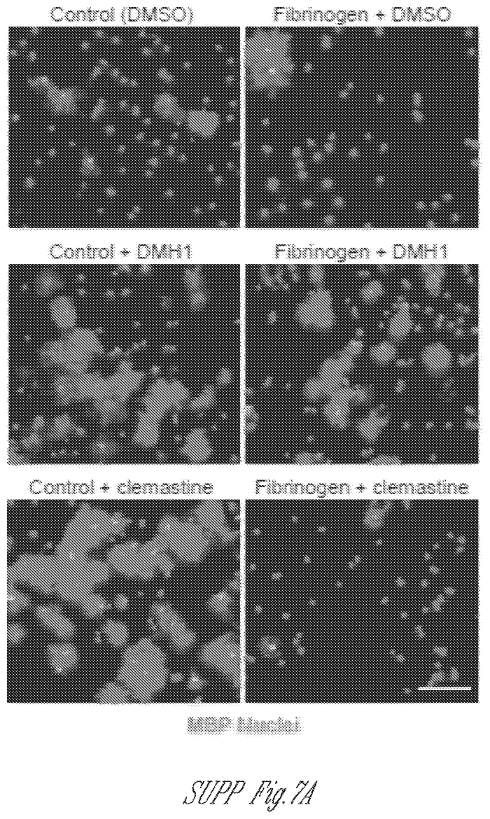

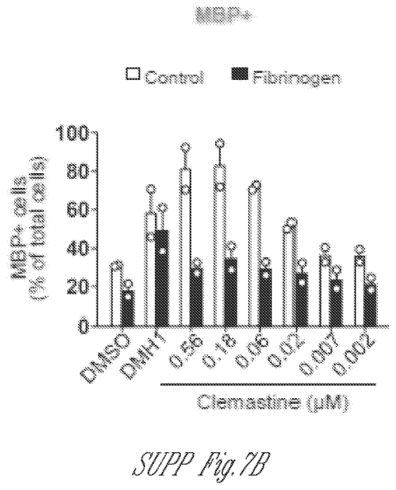

Supplementary FIGS. 4A-B. NG2 cell clusters associated with fibrinogen deposition and myelin disruption at chronic EAE. A, Microscopy of ventral spinal cord sections of NG2-CreER™:Rosa^{tdTomato/+}:Cx3cr1^{GFP/+} mice at chronic EAE immunostained for fibrinogen (green). NG2tdTomato⁺ cells (red) cluster at sites of fibrinogen deposition, depicted here in the merge channel with yellow ROIs (white arrowheads). Scale bar, 50 μm. B, In vivo 2P maximum intensity projection images of NG2^{tdTomato+} cells (red) and myelin (green) in NG2-CreER™:Rosa^{tdTomato/+}:Cx3cr1^{GFP/+} mice at chronic EAE in areas of NG2 cell clusters and areas without clusters. It is important to note that myelin sheathes are labeled with MitoTracker Deep Red far red-fluorescent dye (abs/em ˜644/665 nm), pseudocolored here in green. Disrupted myelin or myelin blebs are shown here with white arrows in areas of NG2 cell clusters and normal-appearing myelin is depicted with white arrowheads in non-cluster areas. Scale bar, 20 μm.

Supplementary FIGS. 5A-C. FACS isolation of NG2 cells. A, Representative flow cytometry plots of the gating strategy for NG2^{tdTomato+} cells from the spinal cord of EAE (n=3) and healthy control mice (n=3) for bulk RNA-seq. B, Representative flow cytometry plots of the gating strategy for PDGFRα⁺ and PDGFRβ⁺ cells from the spinal cord of chronic EAE (n=5) and healthy control mice (n=5) for cell surface staining. C, Representative flow cytometry contour plot and quantification of surface MHCII in live PDGFRα⁺ cells. Data are from n=5 per group (mean±s.e.m.) **p<0.01, (two-tailed Mann-Whitney test). Percent of cell population is listed above gate (A-C).

Supplementary FIGS. 6A-C. Ratio of oligodendroglial lineage cells and pericytes amongst NG2^{tdTomato+} cells in control and Peak EAE. A, Microscopy of ventral spinal cord sections of NG2-CreER™:Rosa^{tdTomato/+} mice in control and at peak EAE with NG2^{tdTomato+} cells (red) immunostained for OLIG2 (green) and PDGFRβ (stained in far red channel, pseudocolored here in blue). NG2^{tdTomato+} OLIG2⁺ cells are depicted with white arrowheads; NG2^{tdTomato+} PDGFRβ⁺ cells are depicted with white asterisks. NG^{2tdTomato+} OLIG2⁻ PDGFRβ⁻ cells are depicted with white arrows. Scale bar, 20 μm. B-C, Quantifications of the percentage of NG^{2tdTomato+} cells that are OLIG2⁺ and PDGFRβ⁺ in control and at peak EAE.

Supplementary FIG. 7A-C. Effect of clemastine on primary OPCs in the presence of fibrinogen. A, Immunofluorescence for MBP (green) in primary rat OPCs treated with fibrinogen and clemastine (0.56 μM), DMH1 (1 μM), or vehicle control (dimethylsulfoxide, DMSO) for three days in differentiation media without T3 or growth factors. Nuclei are stained with Hoechst dye (blue). Representative images from n=2 independent experiments. Scale bar, 100 μm. B, Quantification of percentage of total cells MBP+ from automated image acquisition and quantification. Data are mean±s.e.m. from n=2 independent experiments.

### DETAILED DESCRIPTION OF THE INVENTION

The practice of the methods and compositions described herein may employ, unless otherwise indicated, conventional techniques of pharmaceutical chemistry, drug formulation techniques, dosage regimes, molecular biology and biochemistry, all of which are within the skill of those who practice in the art. Such conventional techniques include the use of combinations of therapeutic regimes including but not limited to the methods described herein; technologies for formulations of adjunct therapies used in combination with known, conventional therapies and/or new therapies for the treatment of neurodegeneration, delivery methods that are useful for the compositions of the invention, and the like.

### Definitions

For the purposes of clarity and a concise description, features can be described herein as part of the same or separate embodiments; however, it will be appreciated that the scope of the invention may include embodiments having combinations of all or some of the features described.

The terminology used herein is for the purpose of describing particular embodiments only and is not intended to be limiting of the invention. Unless defined otherwise, all technical and scientific terms used herein have the same meaning as commonly understood by one of ordinary skill in the art to which this invention belongs. The following definitions are intended to aid the reader in understanding the present invention but are not intended to vary or otherwise limit the meaning of such terms unless specifically indicated.

As used herein, the indefinite articles “a”, “an” and “the” should be understood to include plural reference unless the context clearly indicates otherwise. Thus, for example, reference to “an inhibitor” refers to one or more agents with the ability to inhibit a target molecule, and reference to “the method” includes reference to equivalent steps and methods known to those skilled in the art, and so forth.

The phrase “and/or,” as used herein, should be understood to mean “either or both” of the elements so conjoined, e.g., elements that are conjunctively present in some cases and disjunctively present in other cases.

As used herein, “or” should be understood to have the same meaning as “and/or” as defined above. For example, when separating a listing of items, “and/or” or “or” shall be interpreted as being inclusive, e.g., the inclusion of at least one, but also including more than one, of a number of items, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein, the term “about” means plus or minus 10% of the indicated value. For example, about 100 means from 90 to 110.

Where a range of values is provided, it is understood that each intervening value, between the upper and lower limit of that range and any other stated or intervening value in that stated range is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either both of those included limits are also included in the invention.

A “CNS disorder” can be any disease, disorder or injury associated with the toxicity of a population of cells within the CNS. In one example, the CNS disorder is associated with a pathological process such as neurodegeneration, demyelination, dysmyelination, axonal injury, and/or dysfunction or death of an oligodendrocyte or a neuronal cell, or loss of neuronal synapsis/connectivity. In other examples, the CNS disorder is a disease associated with plaque formation, e.g., amyloid plaque formation. CNS disorders include neurodegenerative disorders that affect the brain or spinal cord of a mammal. In certain embodiments, the CNS disorder has one or more inflammatory components.

The term “neurodegenerative diseases” includes any disease or condition characterized by problems with movements, such as ataxia, and conditions affecting cognitive abilities (e.g., memory) as well as conditions generally related to all types of dementia. “Neurodegenerative diseases” may be associated with impairment or loss of cognitive abilities, potential loss of cognitive abilities and/or impairment or loss of brain cells. Exemplary “neurodegenerative diseases” include Alzheimer's disease (AD), diffuse Lewy body type of Alzheimer's disease, Parkinson's disease, Down syndrome, progressive multiple sclerosis (MS), dementia, mild cognitive impairment (MCI), amyotrophic lateral sclerosis (ALS), traumatic brain injuries, ischemia, stroke, cerebral ischemic brain damage, ischemic or hemorrhaging stroke, multi-infarct dementia, hereditary cerebral hemorrhage with amyloidosis of the Dutch-type, cerebral amyloid angiopathy (including single and recurrent lobar hemorrhages), neurodegeneration induced by viral infection (e.g. AIDS, encephalopathies) and other degenerative dementias, including dementias of mixed vascular and degenerative origin, dementia associated with Parkinson's disease, dementia associated with progressive supranuclear palsy and dementia associated with cortical basal degeneration, epilepsy, seizures, and Huntington's disease.

As used herein, a disease, disorder or condition is “treated” if at least one pathophysiological measurement associated with the disease is decreased and/or progression of a pathophysiological process is reversed, halted or reduced. For example, a disease, disorder or condition can be “treated” if the number of plaques present in the CNS of a patient with a neurodegenerative disease is reduced, remains constant, or the creation of new plaques is slowed by the administration of an agent. In another example, a disease, disorder or condition can be “treated” if one or more symptoms of the disease or disorder is reduced, alleviated, terminated, slowed, or prevented. Measurement of one or more exemplary clinical hallmarks and/or symptoms of a disease can be used to aid in determining the disease status in an individual and the treatment of one or more symptoms associated therewith.

The term “administering” as used herein refers to administering to a subject and/or contacting a neuron or portion thereof with an inhibitor as described herein. This includes administration of the inhibitor to a subject in which the neuron is present, as well as introducing the inhibitor into a medium in which a neuron is cultured. Administration “in combination with” one or more further agents include concurrent and consecutive administration, in any order.

The term “neuron” as used herein denotes nervous system cells that include a central cell body or soma, and two types of extensions or projections: dendrites, by which, in general, the majority of neuronal signals are conveyed to the cell body, and axons, by which, in general, the majority of neuronal signals are conveyed from the cell body to effector cells, such as target neurons or muscle. Neurons can convey information from tissues and organs into the central nervous system (afferent or sensory neurons) and transmit signals from the central nervous systems to effector cells (efferent or motor neurons). Other neurons, designated interneurons, connect neurons within the central nervous system (the brain and spinal column). Certain specific examples of neuron types that may be subject to treatment according to the invention include cerebellar granule neurons, dorsal root ganglion neurons, and cortical neurons.

The terms “mammal” and “mammalian subject” as used herein refers to any animal classified as a mammal, including humans, higher non-human primates, rodents, and domestic and farm animals, such as cows, horses, dogs, and cats. In some embodiments of the invention, the mammal is a human.

The term “pharmaceutical composition” refers to a formulation containing the disclosed compounds in a form suitable for administration to a subject. In one embodiment, the pharmaceutical composition is in bulk or in unit dosage form. The unit dosage form is any of a variety of forms, including, for example, a tablet, capsule, or a vial. The quantity of active ingredient in a unit dose of composition is an effective amount and is varied according to the particular treatment involved.

The phrase “therapeutically effective amount” or “effective amount” used in reference to an agent of the invention is an art-recognized term. In certain embodiments, the term refers to an amount of an agent that produces some desired effect at a reasonable benefit/risk ratio applicable to any medical treatment. In certain embodiments, the term refers to that amount necessary or sufficient to eliminate, reduce or maintain a target of a particular therapeutic regimen. The effective amount may vary depending on such factors as the disease or condition being treated, the particular targeted constructs being administered, the size of the subject or the severity of the disease or condition. One of ordinary skill in the art may empirically determine the effective amount of a particular compound without necessitating undue experimentation.

“Inhibitors,” “activators,” and “modulators” are used to refer to activating, inhibitory, or modulating (increase, inhibit, decrease or activate expression or activity as compared to control (an untreated or healthy subject/mammal) molecules. Inhibitors are compounds that, e.g., bind to, partially or totally block activity, decrease, prevent, delay activation, inactivate, desensitize, or down regulate the activity or expression. “Activators” are compounds that increase, open, activate, facilitate, enhance activation, sensitize, agonize, or up regulate activity, e.g., agonists.

In certain embodiments, a therapeutically effective amount of an agent for in vivo use will likely depend on a number of factors, including: the rate of release of an agent from a polymer matrix, which will depend in part on the chemical and physical characteristics of the polymer; the identity of the agent; the mode and method of administration; and any other materials incorporated in the polymer matrix in addition to the agent. In certain embodiments, a therapeutically effective amount is the amount effective to promote myelination in the subject's central nervous system.

Fibrinogen (coagulation factor I) is a 340-kDa protein secreted by hepatocytes in the liver and present in the blood circulation at 3-5 mg/ml (2, 3). Fibrinogen is cleaved by thrombin and, upon conversion to fibrin, serves as the major architectural protein component of blood clots. In CNS disease fibrinogen enters the CNS in areas with vascular permeability or blood-brain barrier (BBB) disruption and is deposited as insoluble fibrin forming a provisional extracellular matrix during brain repair (3, 4). Fibrin is present in the brain in a wide range of CNS pathologies, such as multiple sclerosis (MS), Alzheimer disease (AD), stroke, and traumatic brain injury (TBI) (3). Fibrinogen acts as a multi-faceted signaling molecule by interacting with integrins and non-integrin receptors and by functioning as a carrier of growth factors regulating their bioavailability (3-7). Thereby fibrinogen promotes inflammation and neurodegeneration, while it inhibits myelin repair (3). However, the role of fibrinogen in NSPC differentiation remains unknown.

As used herein, the terms “including”, “includes”, “having”, “has”, “with”, or variants thereof, are intended to be inclusive similar to the term “comprising.”

As used herein, said “contain”, “have” or “including” include “comprising”, “mainly consist of”, “basically consist of” and “formed of”; “primarily consist of”, “generally consist of” and “comprising of” belong to generic concept of “have” “include” or “contain”.

The terms “comprises,” “comprising,” and the like can have the meaning ascribed to them in U.S. Patent Law and can mean “includes,” “including” and the like. As used herein, “including” or “includes” or the like means including, without limitation.

This Summary is provided to introduce a selection of concepts in a simplified form that are further described below in the Detailed Description. This Summary is not intended to identify key or essential features of the claimed subject matter, nor is it intended to be used to limit the scope of the claimed subject matter. Other features, details, utilities, and advantages of the claimed subject matter will be apparent from the following written Detailed Description including those aspects illustrated in the accompanying drawings and defined in the appended claims.

The present invention provides methods and compositions for treating a neurological disease, disorder or injury. The present invention also provides methods and compositions for preserving or protecting neural structure and/or function in a subject in need thereof, such as in a mammalian subject by administering one or more agents and/or compositions described herein to the subject.

One embodiment provides a method of treating or preventing neurodegeneration in a mammal, such as a human, comprising administering to the mammal in need thereof an effective amount of an inhibitor of at least one bone morphogenetic protein (BMP) receptor.

One embodiment provides a method of treating or preventing neurodegeneration in a mammal, such as a human, comprising administering to the mammal in need thereof an effective amount of a small molecule inhibitor (e.g., compounds that block the receptor) of ACVR1 (Alk2).

One embodiment provides for a method to promote remyelination in neurological diseases or disorders in a mammal, such as a human, comprising administering to the mammal in need thereof an effective amount of a small molecule inhibitor of ACVR1 (Alk2).

Some embodiments provide for methods and compositions for preventing or ameliorating demyelination in a subject, such as mammalian subject, by administering to the mammal in need thereof an effective amount of a small molecule inhibitor of ACVR1 (Alk2).

Other embodiments provide methods and compositions for enhancing myelination and/or re-myelination in a mammalian subject, such as a human subject, by administering to the mammal in need thereof an effective amount of a small molecule inhibitor of ACVR1 (Alk2).

In one embodiment, the small molecule inhibitor of ACVR1 (Alk2) is LDN-212854 or derivatives or variants thereof.

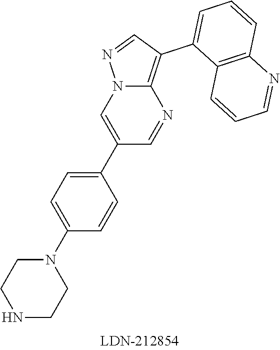 [SMILES: c1cc(-c2cnn3cc(-c4ccc(N5CCNCC5)cc4)cnc23)c2cccnc2c1]

In another embodiment, the small molecule inhibitor of ACVR1 (Alk2 (ALK-2 activin receptor-like kinase 2)) is dorsomorphin or derivatives or variants thereof.

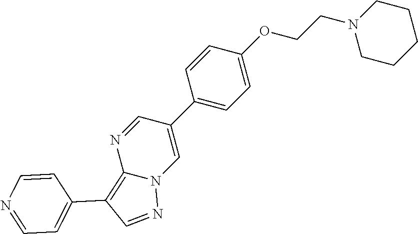 [SMILES: c1cc(-c2cnn3cc(-c4ccc(OCCN5CCCCC5)cc4)cnc23)ccn1]

In another embodiment, the small molecule inhibitor of ACVR1 (Alk2) and/or BMP is DMH1 or derivatives or variants thereof.

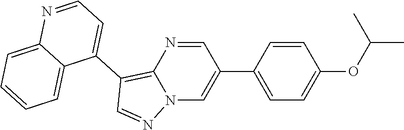 [SMILES: CC(C)Oc1ccc(-c2cnc3c(-c4ccnc5ccccc45)cnn3c2)cc1]

In one embodiment, the small molecule inhibitor of ACVR1 (Alk2) is saracatinib (also known as AZD0530; ifopa.org/saracatinib) or derivatives or variants thereof.

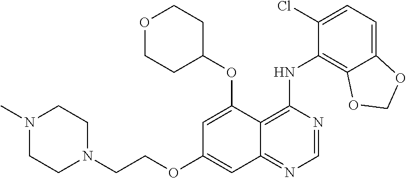 [SMILES: CN1CCN(CCOc2cc(OC3CCOCC3)c3c(Nc4c(Cl)ccc5c4OCO5)ncnc3c2)CC1]

In one embodiment, the small molecule inhibitor of ACVR1 (Alk2) is BCX9250 (ir.biocryst.com/news-releases/news-release-details/biocryst-announces-positive-phase-1-results-bcx9250-oral-alk-2) or derivatives or variants thereof.

In one embodiment, the small molecule inhibitor of ACVR1 (Alk2) is KER-047 (kerostx.com/our-leads) or derivatives or variants thereof.

In one embodiment, the small molecule inhibitor of ACVR1 (Alk2) is INCB000928 (ashpublications.org/blood/article/136/Supplement %201/52/472793/Characterization-of-INCB00928-a-Potent-and) or derivatives or variants thereof.

In one embodiment, the small molecule inhibitor of ACVR1 (Alk2) is BLU-782 (https://www.ipsen.com/press-releases/ipsen-and-blueprint-medicines-announce-exclusive-global-license-agreement-to-develop-and-commercialize-blu-782-for-the-treatment-of-fibrodysplasia-ossificans-progressiva-fop/) or derivatives or variants thereof.

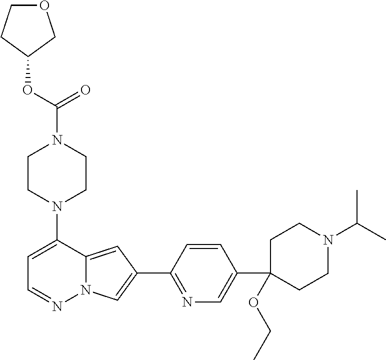 [SMILES: CCOC1(c2ccc(-c3cc4c(N5CCN(C(=O)O[C@@H]6CCOC6)CC5)ccnn4c3)nc2)CCN(C(C)C)CC1]

In another embodiment, the small molecule inhibitor of ACVR1 (Alk2) is momelotinib (sierraoncology.com/momelotinib-overview/) or derivatives or variants thereof.

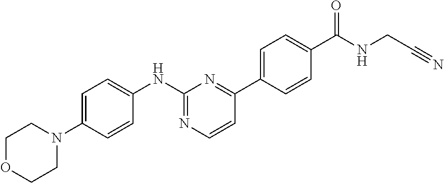 [SMILES: N#CCNC(=O)c1ccc(-c2ccnc(Nc3ccc(N4CCOCC4)cc3)n2)cc1]

In another embodiment, the small molecule inhibitor of ACVR1 (Alk2) is LDN-193189 or derivatives or variants thereof.

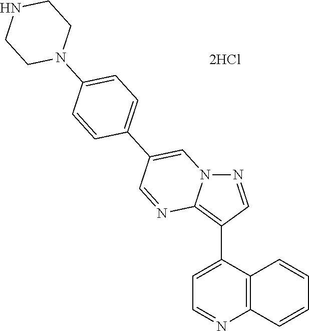 [SMILES: c1ccc2c(-c3cnn4cc(-c5ccc(N6CCNCC6)cc5)cnc34)ccnc2c1]

In another embodiment, the small molecule inhibitor of ACVR1 (Alk2) is K02288 or derivatives or variants thereof.

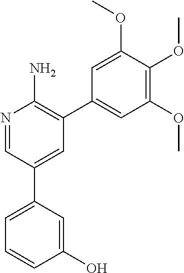 [SMILES: COc1cc(-c2cc(-c3cccc(O)c3)cnc2N)cc(OC)c1OC]

In another embodiment, the small molecule inhibitor of ACVR1 (Alk2) is LDN-214117 or derivatives or variants thereof.

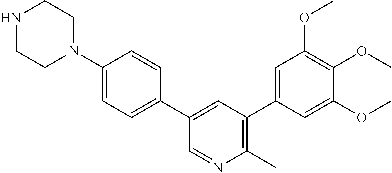 [SMILES: COc1cc(-c2cc(-c3ccc(N4CCNCC4)cc3)cnc2C)cc(OC)c1OC]

In another embodiment, the small molecule inhibitor of ACVR1 (Alk2) is LDN-213844 or derivatives or variants thereof.

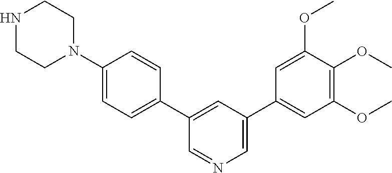 [SMILES: COc1cc(-c2cncc(-c3ccc(N4CCNCC4)cc3)c2)cc(OC)c1OC]

In another embodiment, the small molecule inhibitor of ACVR1 (Alk2) is M4K2009 or derivatives or variants thereof.

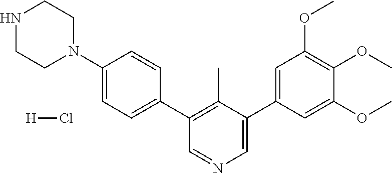

In another embodiment, the small molecule inhibitor of ACVR1 (Alk2) is M4K2149 or derivatives or variants thereof.

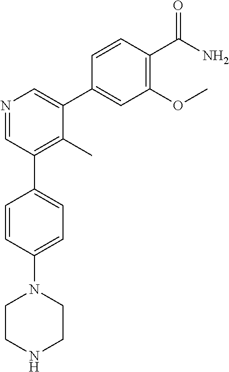 [SMILES: COc1cc(-c2cncc(-c3ccc(N4CCNCC4)cc3)c2C)ccc1C(N)=O]

In one embodiment, the mechanism of action that differentiates these compounds from the promyelinating compounds is that there are effects on inhibition of astrogenesis (astrocyte differentiation from the progenitors). Promyelinating compound will promote myelin formation, but they will not suppress astrogliosis at the same time. ACVR1 inhibition does both. The compounds have dual functions as promoters of remyelination and suppressors of the glial scar.

In one embodiment, said mammal has been diagnosed with a disease, disorder, or injury involving demyelination, dysmyelination, or neurodegeneration. In one embodiment, said disease, disorder, or injury is selected from the group consisting of multiple sclerosis (MS), progressive multifocal leukoencephalopathy (PML), encephalomyelitis (EPL), central pontine myelolysis (CPM), adrenoleukodystrophy, Alexander's disease, Pelizaeus Merzbacher disease (PMZ), Wallerian Degeneration, optic neuritis, transverse myelitis, amyotrophic lateral sclerosis (ALS), Huntington's disease, Alzheimer's disease, Parkinson's disease, spinal cord injury, traumatic brain injury, neonatal brain injury, post radiation injury, neurologic complications of chemotherapy, stroke, acute ischemic optic neuropathy, vitamin E deficiency, isolated vitamin E deficiency syndrome, AR, Bassen-Kornzweig syndrome, Marchiafava-Bignami syndrome, metachromatic leukodystrophy, trigeminal neuralgia, acute disseminated encephalitis, Guillain-Barre syndrome, Marie-Charcot-Tooth disease and Bell's palsy.

One embodiment also includes pharmaceutical compositions and kits that contain one or more agents that can be used to inhibit degeneration of a neuron or a portion thereof, as described herein, such as an inhibitor of ACVR1 (Alk2). The pharmaceutical compositions and kits can optionally include one or more pharmaceutically acceptable excipients.

Another embodiment features a packaged composition (e.g., a packaged pharmaceutical composition) that includes at least one agent disclosed herein that is labeled and/or contains instructions for use of said agent for treating a neurological disease. The agent can be in a form suitable for any route of administration, e.g., oral administration, peripheral administration, intrathecal administration, etc. One or more active agents can be included in the packaged pharmaceutical composition.

Further provided herein is a method to screen for inhibitors of ACVR1 (Alk2) or other bone morphogenetic protein (BMP) receptors.

Currently, remyelinating compounds to overcome extrinsic inhibition of remyelination are not available. The competitive advantage using this compound is to promote remyelination in the presence of inflammation and blood-brain barrier leaks in diseases such as multiple sclerosis (to overcome the fibrinogen inhibitory environment to promote remyelination in chronic neuroinflammation).

LDN-212854 as water soluble ACVR1 inhibitor that can be used in vivo for treatment of neurological disease. For, example, LDN-212854 enhanced formation of mature oligodendrocytes from fibrinogen treated OPCs (in vitro Fibrinogen-OPC differentiation assay). Additionally, LDN-212854 improved clinical scores and reduced spinal cord Id2 protein levels (in vivo PLP-EAE)

Further, ACVR1 BMP receptor inhibitor promotes OL differentiation and blocks astrocyte fate of OPCs. BMP receptor inhibitor improves clinical scores in EAE. BMP receptor inhibition reduces perivascular NG2 cell clusters in EAE. BMP receptor inhibitor reduces myelin pathology in EAE.

NG2 cell-vascular interactions are altered in fibrinogen-rich neuroinflammatory lesions. BMP pathway activation provides a mechanistic link between NG2 cell, vascular and myelin pathology in neuroinflammation. BMP receptor blockade with LDN-212854 restores oligovascular homeostasis and overcomes extrinsic inhibition of remyelination. ACVR1 (ALK2) receptor inhibition to treat neurological diseases.

### Administration

Pharmaceutical formulations of the agents described herein are prepared by combining the agent having the desired degree of purity with optional physiologically acceptable carriers, excipients, or stabilizers (see, e.g., Remington's Pharmaceutical Sciences (18th edition), ed. A. Gennaro, 1990, Mack Publishing Co., Easton, Pa.). Acceptable carriers, excipients, or stabilizers are nontoxic to recipients at the dosages and concentrations employed, and can include buffers such as phosphate, citrate, and other organic acids; antioxidants including ascorbic acid, BHA, and BHT; low molecular weight (less than about 10 residues) polypeptides; proteins, such as serum albumin, gelatin or immunoglobulins; hydrophilic polymers such as polyvinylpyrrolidone, amino acids such as glycine, glutamine, asparagine, arginine, or lysine; monosaccharides, disaccharides, and other carbohydrates including glucose, mannose, or dextrins; chelating agents such as EDTA; sugar alcohols such as mannitol or sorbitol; salt forming counter-ions such as sodium; and/or nonionic surfactants such as Tween, Pluronics, or PEG.

Agents to be used for in vivo administration can be sterile/aseptic, which can be achieved by filtration through sterile filtration membranes, prior to or following lyophilization and reconstitution. Therapeutic compositions may be placed into a container having a sterile access port, for example, an intravenous solution bag or vial.

Agents described herein can be optionally combined with or administered in concert with each other or other agents known to be useful in the treatment of the relevant disease or condition.

Thus, in the treatment of demyelinating diseases, the agents can be administered in combination with other promyelinating drugs, such as clemastine.

Thus, in the treatment of demyelinating diseases, the agents can be administered in combination with injectable compositions including interferon beta la inhibitors or interferon beta lb inhibitors, glatiramer acetate, and daclizumab; oral medications such as teriflunomide, fingolimod, and dimethyl fumarate; or infused medications such as alemtuzumab, mitoxantrone, or natalizumab.

In the treatment of Alzheimer's disease, agents can be administered with acetylcholinesterase inhibitors (e.g., donepezil, galantamine, and rivastigmine) and/or NMDA receptor antagonists (e.g., memantine).

In the treatment of ALS, for example, agents can be administered in combination with Riluzole (Rilutek), minocycline, insulin-like growth factor 1 (IGF-1), and/or methylcobalamin.

In another example, in the treatment of Parkinson's disease, agents can be administered with L-dopa, dopamine agonists (e.g., bromocriptine, pergolide, pramipexole, ropinirole, cabergoline, apomorphine, and lisuride), dopa decarboxylase inhibitors (e.g., levodopa, benserazide, and carbidopa), and/or MAO-B inhibitors (e.g., selegiline and rasagiline).

The combination therapies can involve concurrent or sequential administration, by the same or different routes, as determined to be appropriate by those of skill in the art. The invention also includes pharmaceutical compositions and kits.

The route of administration of the agents is selected in accordance with known methods, e.g., injection or infusion by intravenous, intraperitoneal, intracerebral, intramuscular, intraocular, intraarterial or intralesional routes, topical administration, or by sustained release systems as described below.

For intracerebral use, the agents can be administered continuously by infusion into the fluid reservoirs of the CNS, although bolus injection may be acceptable. The agents can be administered into the ventricles of the brain or otherwise introduced into the CNS or spinal fluid. Administration can be performed by use of an indwelling catheter and a continuous administration means such as a pump, or it can be administered by implantation, e.g., intracerebral implantation of a sustained-release vehicle. More specifically, the agents can be injected through chronically implanted cannulas or chronically infused with the help of osmotic minipumps. Subcutaneous pumps are available that deliver proteins through a small tubing to the cerebral ventricles. Highly sophisticated pumps can be refilled through the skin and their delivery rate can be set without surgical intervention. Examples of suitable administration protocols and delivery systems involving a subcutaneous pump device or continuous intracerebroventricular infusion through a totally implanted drug delivery system are those used for the administration of dopamine, dopamine agonists, and cholinergic agonists to Alzheimer's disease patients and animal models for Parkinson's disease, as described by Harbaugh, J. Neural Transm. Suppl. 24:271, 1987; and DeYebenes et al., Mov. Disord. 2:143, 1987.

Suitable examples of sustained release preparations include semipermeable polymer matrices in the form of shaped articles, e.g., films or microcapsules. Sustained release matrices include polyesters, hydrogels, polylactides (U.S. Pat. No. 3,773,919; EP 58,481), copolymers of L-glutamic acid and gamma ethyl-L-glutamate (Sidman et al., Biopolymers 22:547, 1983), poly (2-hydroxyethyl-methacrylate) (Langer et al., J. Biomed. Mater. Res. 15:167, 1981; Langer, Chem. Tech. 12:98, 1982), ethylene vinyl acetate (Langer et al., Id), or poly-D-(−)-3-hydroxybutyric acid (EP 133,988A). Sustained release compositions also include liposomally entrapped compounds, which can be prepared by methods known per se (Epstein et al., Proc. Natl. Acad. Sci. U.S.A. 82:3688, 1985; Hwang et al., Proc. Natl. Acad. Sci. U.S.A. 77:4030, 1980; U.S. Pat. Nos. 4,485,045 and 4,544,545; and EP 102,324A). Ordinarily, the liposomes are of the small (about 200-800 Angstroms) unilamellar type in which the lipid content is greater than about 30 mol % cholesterol, the selected proportion being adjusted for the optimal therapy.

A therapeutically effective amount of an agent will depend, for example, upon the therapeutic objectives, the route of administration, and the condition of the patient. Accordingly, it will be necessary for the therapist to titer the dosage and modify the route of administration as required to obtain the optimal therapeutic effect. A typical daily dosage might range from, for example, about 1 μg/kg to up to 100 mg/kg or more (e.g., about 1 μg/kg to 1 mg/kg, about 1 μg/kg to about 5 mg/kg, about 1 mg/kg to 10 mg/kg, about 5 mg/kg to about 200 mg/kg, about 50 mg/kg to about 150 mg/mg, about 100 mg/kg to about 500 mg/kg, about 100 mg/kg to about 400 mg/kg, and about 200 mg/kg to about 400 mg/kg), depending on the factors mentioned above. Typically, the clinician will administer an active inhibitor until a dosage is reached that results in improvement in or, optimally, elimination of, one or more symptoms of the treated disease or condition. The progress of this therapy is easily monitored by conventional assays. One or more agent provided herein may be administered together or at different times (e.g., one agent is administered prior to the administration of a second agent). One or more agent may be administered to a subject using different techniques (e.g., one agent may be administered orally, while a second agent is administered via intramuscular injection or intranasally). One or more agent may be administered such that the one or more agent has a pharmacologic effect in a subject at the same time. Alternatively, one or more agent may be administered, such that the pharmacological activity of the first administered agent is expired prior the administration of one or more secondarily administered agents.

One skilled in the art, upon reading the present specification, will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, intranasal, inhalational, and the like. Dosage forms for the topical or transdermal administration of a compound described herein includes powders, sprays, ointments, pastes, creams, lotions, gels, solutions, patches, nebulized compounds, and inhalants. In a preferred embodiment, the active compound is mixed under sterile conditions with a pharmaceutically acceptable carrier, and with any preservatives, buffers, or propellants that are required.

The present invention also provides a therapeutic kit containing materials useful for the treatment or prevention of the disorders and conditions described above is provided. The therapeutic kit may include a container and a label or package insert on or associated with the container. Suitable containers include, for example, bottles, vials, syringes, etc. The containers may be formed from a variety of materials such as glass or plastic. The container holds a pharmaceutical composition that is by itself or when combined with another agent effective for treating or preventing the condition and may have a sterile access port (e.g., an intravenous solution bag or a vial having a stopper pierceable by a hypodermic injection needle). At least one active agent in the pharmaceutical composition is one of the agents described herein above. The label or package insert indicates that the composition is used for treating the condition of choice. Moreover, the kit may include (a) a first container with a pharmaceutical composition contained therein, wherein the composition includes an agent described herein; and (b) a second container with a pharmaceutical composition contained therein, wherein the composition includes a different agent. The therapeutic kit in this embodiment of the invention may further include a package insert indicating that the compositions can be used to treat a particular condition. Alternatively, or additionally, the therapeutic kit may further include a second (or third) container including a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### Assessment of Treatment

In some embodiments, the successful treatment of a subject with an agent described herein is determined by at least about a 10%-100% decrease in one or more symptoms of a CNS disorder. Examples of such symptoms include, but are not limited to, slowness of movement, loss of balance, depression, decreased cognitive function, short-term memory loss, long-term memory loss, confusion, changes in personality, language difficulties, loss of sensory perception, sensitivity to touch, numbness in extremities, tremors, ataxia, muscle weakness, muscle paralysis, muscle cramps, muscle spasms, significant changes in eating habits, excessive fear or worry, insomnia, delusions, hallucinations, fatigue, back pain, chest pain, digestive problems, headache, rapid heart rate, dizziness, and visual changes.

For example, clinical signs of MS are routinely classified and standardized, e.g., using an EDSS rating system based on neurological examination and long-distance ambulation. As used herein, the “Expanded Disability Status Scale” or “EDSS” is intended to have its customary meaning in the medical practice. EDSS is a rating system that is frequently used for classifying and standardizing MS. The accepted scores range from O (normal) to 10 (death due to MS). Typically, patients having an EDSS score of about 4-6 will have moderate disability (e.g., limited ability to walk), whereas patients having an EDSS score of about 7 or 8 will have severe disability (e.g., will require a wheelchair). More specifically, EDSS scores in the range of 1-3 refer to an MS patient who is fully ambulatory, but has some signs in one or more functional systems; EDSS scores in the range higher than 3 to 4.5 show moderate to relatively severe disability; an EDSS score of 5 to 5.5 refers to a disability impairing or precluding full daily activities; EDSS scores of 6 to 6.5 refer to an MS patient requiring intermittent to constant, or unilateral to bilateral constant assistance (cane, crutch or brace) to walk; EDSS scores of 7 to 7.5 means that the MS patient is unable to walk beyond five meters even with aid, and is essentially restricted to a wheelchair; EDSS scores of 8 to 8.5 refer to patients that are restricted to bed; and EDSS scores of 9 to 10 mean that the MS patient is confined to bed, and progressively is unable to communicate effectively or eat and swallow, until death due to MS.

In certain embodiments, the evaluation of disease progression includes a measure of upper extremity function (e.g., a 9HP assessment). Alternatively, or in combination, disease progression includes a measure of lower extremity function. Alternatively, or in combination, disease progression includes a measure of ambulatory function, e.g., short distance ambulatory function (e.g., T25FW). Alternatively, or in combination, disease progression includes a measure of ambulatory function, e.g., longer distance ambulatory function (e.g., a 6-minute walk test). In one embodiment, the disease progression includes a measure of ambulatory function other than EDSS ambulatory function. In one embodiment, disease progression includes a measure of upper extremity function e.g., a 9HP assessment, and a measure of ambulatory function, e.g., short distance ambulatory function (e.g., T25FW). In one embodiment, disease progression includes a measure of upper extremity function (e.g., a 9HP assessment) and a measure of lower extremity function. In one embodiment, disease progression includes a measure of upper extremity function (e.g., a 9HP assessment), a measure of lower extremity function, and a measure of ambulatory function, e.g., short distance ambulatory function (e.g., T25FW) and/or longer distance ambulatory function (e.g., a 6-minute timed walk test (e.g., 6MWT)). In one embodiment, one, two or the combination of the T25FW, 6MWT and 9HP assessments can be used to acquire a disease progression value. The measure of ambulatory function (e.g., short distance ambulatory function (e.g., T25FW) or longer distance ambulatory function (e.g., a timed (e.g., 6-minute) walk test (e.g., 6MWT)) and/or measure of upper extremity function (e.g., a 9HP assessment) can further be used in combination with the EDSS to evaluate MS, e.g., progressive forms of MS.

Alzheimer's disease (AD) is a neurodegenerative disorder that results in the loss of cortical neurons, especially in the associative neocortex and hippocampus which in turn leads to slow and progressive loss of cognitive functions, ultimately leading to dementia and death. Major hallmarks of the disease are aggregation and deposition of misfolded proteins such as aggregated beta-amyloid peptide as extracellular senile or neuritic ‘plaques’, and hyperphosphorylated tau protein as intracellular neurofibrillary tangles.

Genetic predispositions for AD are divided into two forms: early-onset familial AD (<60 years), and late-onset sporadic AD (>60 years). Rare, disease causing mutations in Amyloid precursor protein (APP), Presenilin 1 (PSEN1), and Presenilin 2 (PSEN2) genes are known to result in early-onset familial AD while, APOE (allele 4) is the single most important risk factor for late-onset AD. In specific embodiments, the methods of the invention are used to treat subjects with a genetic predisposition for wither early onset familial AD or late-onset sporadic AD.

Although Alzheimer's disease develops differently for every individual, there are many common symptoms. In the early stages, the most common symptom is difficulty in remembering recent events. As the disease advances, symptoms can include confusion, irritability, aggression, mood swings, trouble with language, and long-term memory loss.

Clinical Decision Support Systems (CDSS) comprising computer hardware, software, and/or systems can be used to determine a diagnosis for a patient who has certain symptoms associated with Alzheimer's disease. CDSS often include at least three component parts: a knowledge basis, an inference engine, and a communication mechanism. The knowledge base may comprise compiled information about symptoms, pharmaceuticals, and other medical information. The inference engine may comprise formulas, algorithms, etc. for combining information in the knowledge base with actual patient data. The communication mechanism may be ways to input patient data and to output helpful information based on the knowledge base and inference engine. For example, information may be inputted by a physician using a computer keyboard or tablet and displayed to the physician on a computer monitor or portable device.

In certain aspects, the assessment of treatment includes radiological assessment, e.g., single photon emission computed tomography (SPECT), Positron Emission Tomography (PET), Magnetic Resonance Imaging (MRI) and scintigraphy. For example, multiple sclerosis can be assessed using radiologic assessment of CNS plaques, e.g., by MRI. In another example, AD plaque load can be assessed, e.g., using Aβ-PET.

The assessment of treatment according to the present invention may also be performed using scanning database systems and methods such as those described in US Appln. No. 20150039346.

### BIBLIOGRAPHY

1. Chaker, Z., Codega, P. & Doetsch, F. A mosaic world: puzzles revealed by adult neural stem cell heterogeneity. Wiley Interdiscip. Rev. Dev. Biol. 5, 640-658 (2016).
2. Adams, R. A., Passino, M., Sachs, B. D., Nuriel, T. & Akassoglou, K. Fibrin mechanisms and functions in nervous system pathology. Mol. Inter. 4, 163-176 (2004).
3. Petersen, M. A., Ryu, J. K. & Akassoglou, K. Fibrinogen in neurological diseases: mechanisms, imaging and therapeutics. Nat. Rev. Neurosci. 19, 283-301 (2018).
4. Schachtrup, C. et al. Fibrinogen inhibits neurite outgrowth via beta 3 integrin mediated
5. phosphorylation of the EGF receptor. Proc. Natl Acad. Sci. USA 104, 11814-11819 (2007).
6. Schachtrup, C. et al. Fibrinogen triggers astrocyte scar formation by promoting the availability of active TGF-beta after vascular damage. J. Neurosci. 30, 5843-5854 (2010).
7. Martino, M. M., Briquez, P. S., Ranga, A., Lutolf, M. P. & Hubbell, J. A. Heparin-binding domain of fibrin(ogen) binds growth factors and promotes tissue repair when incorporated within a synthetic matrix. Proc. Natl Acad. Sci. USA 110, 4563-4568 (2013).

### Example

The following example is put forth so as to provide those of ordinary skill in the art with a complete disclosure and description of how to make and use the present invention and is not intended to limit the scope of what the inventors regard as their invention, nor is the example intended to represent or imply that the experiments below are all of or the only experiments performed. It will be appreciated by persons skilled in the art that numerous variations and/or modifications may be made to the invention as shown in the specific aspects without departing from the spirit or scope of the invention as broadly described. The present aspects are, therefore, to be considered in all respects as illustrative and not restrictive.

Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees centigrade, and pressure is at or near atmospheric.

### BMP Receptor Blockade Overcomes Extrinsic Inhibition of Remyelination and Restores Neurovascular Homeostasis

Introduction

Regeneration of CNS myelin fails in several neurological diseases, such as multiple sclerosis, neonatal brain injury, and stroke (Franklin and Ffrench-Constant, 2017). In these conditions, cell-extrinsic cues in the microenvironment inhibit remyelination by blocking multipotent OPCs from differentiating into mature, myelin-producing oligodendrocytes (OLs) (Forbes and Gallo, 2017). A critical barrier to therapeutic advances in chronic demyelinating diseases like multiple sclerosis is the inability to overcome this inhibitory lesion environment and halt disease progression (Reich et al., 2018). Small molecules that enhance intrinsic pathways of OPC differentiation and remyelination have been identified in drug screens (Fancy et al., 2011; Deshmukh et al., 2013; Mei et al., 2014; Najm et al., 2015; Mei et al., 2016). However, these drugs have failed to overcome disease-relevant extrinsic inhibitors of OPC differentiation such as chondroitin sulfate proteoglycans (CSPGs) and inflammatory cytokines and fail to promote OL differentiation in aged OPCs or OPCs from multiple sclerosis patients in an inflammatory environment (Keough et al., 2016; Neumann et al., 2019; Starost et al., 2020). Whether promyelinating compounds can overcome the inhibitory microenvironment at sites of increased vascular permeability remains unknown.

In multiple sclerosis, blood-brain barrier (BBB) disruption allows the blood coagulation factor fibrinogen to enter the CNS (Petersen et al., 2018). Fibrinogen deposition is one of the earliest events in multiple sclerosis pathogenesis and persists in chronically demyelinated lesions but is minimal in remyelinated lesions and absent in normal white matter (Vos et al., 2005; Petersen et al., 2017; Lee et al., 2018). In progressive multiple sclerosis, fibrinogen is detected in the cortex and cerebrospinal fluid and correlates with neuronal and cortical loss (Yates et al., 2017; Magliozzi et al., 2019). In demyelinating injury models, genetic or pharmacologic depletion of fibrinogen promotes remyelination in the CNS and peripheral nervous system (Akassoglou et al., 2002; Petersen et al., 2017). Fibrinogen activates BMP receptor signaling in OPCs and neural precursor cells to inhibit remyelination and neurogenesis, respectively (Petersen et al., 2017; Pous et al., 2020). Fibrinogen induces a cell fate switch of NG2+ (encoded by CSPG-4) OPCs to astrocytes via BMP receptor activation (Petersen et al., 2017), suggesting a role for fibrinogen in extrinsic inhibition of remyelination by inducing OPC-derived astrogenesis in the neurovascular niche. Furthermore, when fibrinogen is converted to fibrin, it induces oxidative stress and pro inflammatory polarization of microglia and macrophages (Ryu et al., 2015; Mendiola et al., 2020), which is toxic to OPCs and contributes to remyelination failure (Back et al., 1998; Miron et al., 2013). This suggests a role for increased vascular permeability and fibrinogen deposition in the maintenance of an inhibitory microenvironment in chronic neurological diseases. However, the remodeling of the neurovascular niche at sites of BBB disruption and its relationship with remyelination failure remains poorly understood.

Here, it is shown that extrinsic inhibition of remyelination by fibrinogen activates signaling pathways in OPCs that could not be overcome by known promyelinating compounds, such as clemastine. In contrast, inhibition of BMP signaling rescued the inhibitory effects of fibrinogen on remyelination by restoring the cell fate of OPCs to mature OLs with therapeutic effects in chronic EAE models. By integrating transcriptomics with in vivo two-photon (2P) imaging co-registered with electron microscopy in chronic neuroinflammatory lesions, it is shown that OPCs accumulate at sites of fibrinogen deposition with active BMP signaling and limited remyelination. Thus, known promyelinating compounds do not overcome BMP receptor activation and abortive OPC differentiation by fibrinogen, suggesting that BMP pathway inhibition may enhance the regenerative potential of the promyelinating progenitor niche at sites of cerebrovascular damage.

Materials and Methods

### Animals

C57BL/6, NOD, B6.Cg-Tg(Cspg4-cre/Esr1*)BAkik/J (NG2-CreER™),¹ B6.Cg-Gt(ROSA)26^{Sortm14(CAG-tdTomato)Hze}/J (Rosa^{tdTomato}),² and B6.129P-Cx3cr1^{tm1Liu}/J (CX3CR1^{GFP})³ mice were purchased from the Jackson Laboratory. Mice were housed in groups of five per cage under standard vivarium conditions and a 12-h light/dark cycle. Sprague-Dawley female rats with litters were purchased from Charles River, and P1-P7 male and female rats were used for OPC isolations. All animal protocols were approved by the Committee of Animal Research at the University of California, San Francisco, and in accordance with the National Institutes of Health and ARRIVE guidelines.

### EAE Induction and Clinical Scoring

Active EAE was induced in 9- to 10-week-old NG2-CreER™:Rosa^{tdTomato/+}:Cx3cr1^{GFP/+} female mice 35-40 days after the last tamoxifen injection by subcutaneous immunization with 75 μg MOG₃₅₋₅₅ peptide (MEVGWYRSPFSRVVHLYRNGK; Auspep), in incomplete Freund's Adjuvant (Sigma-Aldrich) supplemented with 400 μg of heat-inactivated Mycobacterium tuberculosis H37Ra (Difco Laboratories). At day 0 and 2 after immunization, mice were given intraperitoneal injection of 200 ng pertussis toxin (Sigma-Aldrich). For the chronic NOD EAE model, 10- to 12-week-old NOD mice were immunized with 150 μg MOG₃₅₋₅₅ peptide, followed by administration of 200 ng pertussis toxin on days 0 and 2 as described.⁴

For therapeutic treatment, at peak+2d mice were administered 6 mg/kg LDN-212854 (Axon Medchem #2201) or saline twice daily (10-14 hrs apart) for 14 days. Mice were randomly assigned to treatment groups, scored and drug-treated in a blinded manner. Experimental groups were unblinded to treatment assignment at the end of the experiments to ensure experimenter bias was not introduced. Mice that did not develop symptoms of EAE were excluded from treatment and analysis. Mice were weighed and scored daily. Neurological deficits were assessed on a five-point scale by observers blinded to treatment: 0, no symptoms; 1, loss of tail tone; 2, ataxia; 3, hindlimb paralysis; 4 hindlimb and forelimb paralysis; 5, moribund. EAE peak was defined by score >2.5.

### Fluorescence-Activated Cell Sorting of NG2 Cells

For sorting NG2 cells, spinal cord tissues were collected from perfused female mice as previously described.⁵ Single-cell suspensions were prepared from entire spinal cords following the adult brain dissociation (ABD) kit manufacturer's instructions with modification (Miltenyi Biotec). Briefly, minced tissues were individually incubated with ABD Mix 1 containing 15 μM actinomycin D (ActD; Sigma)⁶ for 15 min at 34° C., and then ABD Mix 2 was added to the solution for 10 min at 34° C. Tissues were gently triturated and then incubated for 10 min at 34° C. Homogenized tissue solutions were passed through 70-μm smartstrainer (Miltenyi Biotec), washed with cold Dulbecco's phosphate-buffered saline and centrifuged at 450×g for 7 min at 4° C. Tissue debris was removed following the ABD Kit debris removal step instructions and then passed through 30-μm smartstrainer (Miltenyi Biotec) and centrifuged at 450×g for 7 min at 4° C. All steps above were performed in the presence of 3 μM ActD. Single-cell suspensions were incubated with 1 μM Sytox blue live/dead stain (Thermo Fisher Scientific) for 5 min at 4° C. and then cell sorting was performed on an FACSARIA II (BD Biosciences) with BD FACSDiva™ v8 software. All cells were gated based on SSC-A and FSC-A size and then doublet discrimination was performed by FSC-H and FSC-W parameters. Sytox blue⁻ NG2^{tdtomato+} cells were sorted directly into tubes containing RLT plus lysis buffer (Qiagen) supplemented with 1% 2-mercaptoenthanol (Sigma) and 0.25% reagent DX (Qiagen). Cell lysates were frozen on dry ice and immediately stored at −80° C. until use. For determination of TFPI and MHC class II expression, single cell suspension of C57BL/6 spinal cord tissues were prepared as above without adding ActD. Cells were incubated with Fc Block (BioLegend) for 15 min on ice followed by fluorescently conjugated Abs and anti-TFPI in FACS staining buffers (BD) for 30 min on ice. Cells were then stained with aqua live/dead staining kit (Thermo Fisher Scientific) along with fluorescently conjugated secondary antibody in PBS on ice for 30 min. Samples were run on the LSRFortessa (BD Biosciences) immediately with BD FACSDiva™ v8 software. All FACS plots were generated with Flowjo. Following antibodies were used: APC/Cy7 anti-mouse CD11b (BioLegend, #101225, 1:200), PE anti-mouse CD3 (BioLegend, #100206, 1:200), PE/Cy7 anti-mouse PDGFRA (Invitrogen, #25-1401-82, 1:50), Alexa Fluor 488 anti-mouse PDGFRB (Invitrogen, 53-1402-82, 1:50), BV650 anti-mouse MHCII (BD, #743873, 1:200), rabbit anti-mouse TFPI (Invitrogen, PA5-34578, 1:100), BV421 Donkey anti-rabbit IgG (Biolegend, 406410, 1:200) and LIVE/DEAD™ fixable aqua dead cell stain kit (Invitrogen, L34957, 1:500).

### Bulk RNA Sequencing

Frozen NG2 cell lysates in RLT buffer were thawed at 24° C. and then lysed using the QIAshredder (Qiagen) following manufacturer's instructions. Total RNA was isolated from cell lysates using the RNAeasy micro kit without modification (Qiagen). RNA quality and quantity were determined by Bioanalyzer pico chip analysis (Agilent) and all samples with RNA integrity number >8 were used for RNA-seq library preparation. cDNA libraries were generated from total RNA using the Ovation RNA-seq System V2 (NuGEN). Libraries were quantified and quality checked by KAPA qPCR (Roche) and Bioanalyzer DNA chip analysis (Agilent), respectively. Libraries were pooled and paired-end 75 base pair read length sequenced across 8 lanes on a Nextseq500 (Illumnia), for a sequencing depth of >40 million reads per library. FASTQ files were generated in Biospace following manufacturer's guidelines (Illumina).

### Analysis of Bulk RNA Sequencing

For each sample, read 1 and read 2 FASTQ files were separately catenated, and Illumnia adaptors were trimmed and FASTQ files were quality checked using FASTQC. Next, sequencing reads were aligned to mouse reference genome mm10 with STAR and then counts per gene were quantified by featureCounts as previously described.⁵ DEGs were identified by EdgeR (version 3.24.3),⁷ using cutoffs of log 2 fold change of >1 or <−1 and false discovery rate (FDR) p-value <0.05. Using R (version 3.5.0), K-means HOPACH (version 2.42.0) clustering analysis of DEGs was visualized using pheatmap package (version 1.0.12) and volcano plots were generated with ggplot2 package (version 3.2.1).

### Functional Enrichment and Gene Network Analysis

Functional enrichment analysis of DEGs clustered by HOPACH was performed in Metascape using default parameters,⁸ and significant gene ontology (GO) terms were identified by FDR p-value <0.05. Using RNA-seq normalized counts per million dataset, gene network analyses were performed with GSEA with molecular signatures database biological process for GO (C5.bp.v7.1symbols.gmt) using default settings.^{9, 10} GO terms with p-value <0.10 were used for Enrichment Map Visualization using Cytoscape (version 3.7.2)¹¹ and were unbiasedly clustered using the plugin AutoAnnotate (version 1.3.2) with default settings.

### In Vivo Multiphoton Microscopy

An Ultima IV 2P microscope (Prairie Technologies/Bruker) equipped with a Mai Tai eHP DeepSee and an Insight X3 Ti:sapphire femtosecond laser (pulse width <120 fs, tuning range 690-1040 nm (Mai Tai) and 680-1300 nm (Insight X3), repetition rate 80 MHz; Spectra-Physics/Newport) was used. The lasers were tuned to an excitation wavelength of 910-1150 nm depending on the fluorophore(s). Imaging was performed ˜80-120 μm below the dura mater using an Olympus 25×1.05 NA with 1.6 zoom or a Nikon 10×0.4 NA water-immersion lenses with either a 1.0-1.5-μm or a 3-4-μm z-step, for 40× or 10× magnification respectively. The maximum laser power exiting the objective was <40 mW during all imaging experiments. An IR-blocking filter and 560-nm dichroic were placed in the primary emission beam path before the non-descanned detectors. A 660-nm dichroic and a 692/24-nm+607/45-nm bandpass filter were used to separate MitoTracker Red/far red and tdTomato/rhodamine fluorescence emission, respectively; a 520-nm dichroic and a 542/27-nm+494/41-nm bandpass filter were used to separate YFP and GFP fluorescence emission, respectively.

### In Vivo Spinal Cord Imaging

In vivo spinal cord imaging was performed as previously described.¹² Briefly, the spinal cord was exposed at the desired level (T11) through a single laminectomy, and mice were positioned on a spinal stabilization device. Flow-It® ALC (Pentron) was used to create a well around the exposed spinal cord and a drop of pre-warmed (37° C.) artificial cerebrospinal fluid (ACSF, HEPES-based; in mM: 125 NaCl, 10 glucose, 10 HEPES, 3.1 CaCl2, 2.7 KCl, and 1.3 MgCl2; pH 7.4) was applied, preceded by gentle flushing of the dura mater with pre-warmed ACSF to clean and remove potential dural bleedings. Mice were excluded from the study if they sustained accidental injury during the laminectomy or there were signs of (sub-) dural hemorrhage, as these events would cause inflammatory and other neurodegenerative responses unrelated to the experimental design. A 100-μl solution of 3% 70-kDa Oregon green-conjugated dextran (Thermo Fisher Scientific) in ACSF was injected retro-orbitally to label the vasculature, after which the mouse was placed underneath the 2P imaging microscope. For in vivo myelin imaging, the meninges (dura mater and arachnoidea) were carefully removed using a hypodermic needle and the underlying exposed spinal cord was bathed with MitoTracker Deep Red (Thermo Fisher Scientific) dissolved in ACSF at a concentration of 8 μM for 30 min.¹³ The spinal cord was then carefully washed 4-5 times with pre-warmed ACSF before the imaging session.

### Processing of In Vivo Imaging Data

To generate images for figures, z-stacks were intensity-projected along the z-axis using the ImageJ (NIH) summation projection algorithm to recreate two-dimensional representations of the imaged volumes. Images were adjusted for brightness/contrast, background noise and sharpness with ImageJ using Subtract Background, Remove Outliers and Unsharp mask algorithms. The spectral unmixing algorithm in ImageJ was used to separate the GFP and YFP signals, which were subsequently pseudocolored.

### Quantification of Cell Clusters

Z-stacks of images from NG2-CreER™:Rosa^{tdTomato/+}:Cx3cr1^{GFP/+} healthy control or EAE-challenged mice were z-projected and automatically thresholded (default algorithm of ImageJ), to account for signal intensity differences between experiments. NG2 and microglial clusters were defined as areas where 4 or more cell bodies were touching each other, and cell density was at least two-fold higher than in healthy appearing spinal cord. Cluster number and distance to the closest blood vessel were measured with ImageJ.

Myelin Circularity Myelin damage was quantified with myelin circularity. A value of 1.0 indicates a perfect circle (as seen in degenerating myelin in longitudinal sections); as the value approaches 0.0, it indicates an increasingly noncircular, linear shape (longitudinal section of normal myelinated fiber).

### Electron Microscopy

Tissue Preparation for SBLEM. In vivo 2P imaging of NG2-CreER™:Rosa^{tdTomato/+}:Cx3cr1^{GFP/+} mice was performed at chronic EAE to reveal tdTomato+ NG2-lineage cells, microglia, and the vasculature visualized with dextran. After the imaging session, the animal was perfused with Ringers solution followed by 0.5% glutaraldehyde/2% PFA in cacodylate. The region of spinal cord under the imaging window was cut from the perfused cord and post-fixed for 2 hours in cold 0.5% glutaraldehyde/2% PFA in cacodylate. The specimen was then post-fixed overnight in cold 4% PFA in cacodylate. The dorsal aspect of the cord was cut into 150 μm thick horizontal vibratome sections. The sections were post-fixed overnight in cold 2% glutaraldehyde in cacodylate. The sections were stained as previously described.¹⁴ Briefly, the tissue was stained with 2% osmium tetroxide (Ted Pella) in 0.15M cacodylate, 0.5% aq. thiocarbohydrazide (Electron Microscopy Sciences), 2% aq. osmium tetroxide, 2% aq. uranyl acetate (Ted Pella), and lead aspartate,¹⁵ with thorough washing with water between each staining solution. The sections were then dehydrated through ethanol and acetone and then infiltrated with Durcupan ACM (Millipore Sigma). The sections were flat-embedded between glass slides coated with mold-release compound (Electron Microscopy Sciences, Hatfield Pa.) and cured at 60° C. for 72 hours.

X-ray Microscopy and ROI Targeting for SBEM. Specimens were imaged with XRM in order to find and orient ROIs for SBEM imaging¹⁶ Specimens were scanned with a Zeiss Versa 510. Initial scans of whole vibratome slices were collected with a 0.4× objective at 80 kV and a pixel size of approximately 5 μm. After comparison of the vasculature observed in the XRM and two-photon volumes, the ROI was identified and cut out using a razor blade. The specimens were glued onto a piece of ACLAR (Ted Pella), itself glued to a dummy block, using cyanoacrylate glue, with the ventral aspect of the vibratome slice facing up. Using the XRM volume as guidance, the specimen was approached with a glass blade on a Leica EM UC6 ultramicrotome so that the cutting plane was parallel with the desired final cutting plane in the SBEM. Once excess epoxy was removed and tissue exposed, the specimen was removed from the dummy block and attached to an A3 SBEM specimen pin (RMC Boeckeler) using conductive silver epoxy (Ted Pella), this time with the dorsal aspect facing up. The A3 pin was placed in the A3 specimen holder and scanned with XRM using the 4× objective at 80 kV for a pixel size of approximately 1.5 μm. This XRM volume was used to precisely adjust the tilt of the specimen block, remove excess resin from the dorsal aspect of the block, and identify the ROI location for SBEM imaging.

SEM Imaging. Specimens were imaged on a Zeiss Gemini 300 VP SEM equipped with a focal charge compensation system and a Gatan 2XP 3View system. Volumes were collected at 2.5 kV with 1 μsec dwell time, 10 nm pixels, 50 nm step size, and focal gas injection with nitrogen gas turned on. The scope was run in analytic mode and high current mode. The resulting stacks of images were aligned using a custom Python script using IMOD programs.¹⁷

### OPC-X-Screen

Primary rat O4⁺ OPCs were isolated as previously described by immunopanning papain-dissociated cortical cell suspensions sequentially on three dishes: RAN-2 (negative selection), O1 (negative selection), and O4 (positive selection).¹⁸ O4+ OPCs were seeded on polyethyleneimine (PEI, Sigma-Aldrich)-coated 10 cm culture plate at an initial density of 5×10⁵ cells per plate and expanded in proliferation media for 3 days in a 5% CO2 incubator at 37° C. Cells were then passaged using Accutase and re-plated into PEI-coated μClear® 96 well plates (Greiner Bio-One) at 5×10³ cells per well. Cells were incubated in proliferation media for 1 day prior to experimental treatments which were performed in differentiation media. The chemically defined base media was DMEM (4.5 g/L glucose, +pyruvate, +glutamine; Thermo Fisher Scientific), 1× B27 (Thermo Fisher Scientific), 1× N2 (Thermo Fisher Scientific), 1% penicillin-streptomycin (Thermo Fisher Scientific), and 50 ng ml⁻¹ NT3 (Peprotech). Proliferation media consisted of the base media supplemented with 20 ng ml⁻¹ PDGF-AA (Peprotech). Differentiation media consisted of the base media supplemented with 20 ng ml⁻¹ CNTF (Peprotech) and 40 ng ml⁻¹ triiodothyronine (T3, Sigma-Aldrich) with no PDGF-AA. “Slow” differentiation media (base media with no NT3 or additional growth factors and no T3) was used in clemastine dose-response studies to recapitulate the conditions in previous reports.¹⁹

To mimic the inhibitory lesion environment, fibrinogen (Millipore Sigma) was added to differentiation media at a concentration of 1.5 mg ml⁻¹ for the myelin-promoting compound screen and 2.5 mg ml⁻¹ for all other in vitro studies, which are physiologic plasma concentrations known to inhibit OPC differentiation to mature OLs.¹⁸ Myelin-promoting compounds were dissolved in DMSO and added to quadruplicate wells at a concentration previously shown to promote OPC differentiation to OLs 1 hour before fibrinogen treatment. Final compound concentrations were: benztropine 1 μM,¹⁹ clemastine 1 μM,¹⁹ quetiapine 1 μM,¹⁹ miconazole 1 μM,²⁰ clobetasol 5 μM,²⁰ (±)U-50488 1 μM,²¹ and XAV-939 0.1 μM²². DMH1 (1 μM)¹⁸ served as a positive control in all assay plates. Cells were exposed to a maximum DMSO concentration of 0.1%, and controls contained an equal concentration of DMSO. All conditions were tested in quadruplicate wells and repeated in three independent experiments for an N=3 biological replicates. For dose response curves, LDN-212854 and clemastine were added to quadruplicate wells in three-fold serial dilutions (5 μM to 2 nM) 1 hour prior to fibrinogen treatment. Dose-response experiments were repeated in two or three independent experiments. Cells were allowed to differentiate for 3 days prior to fixation, staining, and quantification. For testing the combination of a BMP receptor inhibitor and another promyelinating compound, LDN-212854 (0.1 μM) and clemastine (0.5 μM) were added alone or together in quadruplicate wells 1 hour before fibrinogen treatment in three independent experiments. Cells were allowed to differentiate for 2 days prior to fixation, staining, and quantification.

OPCs were fixed with 4% paraformaldehyde, blocked and permeabilized in 5% normal goat serum/0.1% Triton-X100, and stained with 2 μg/mL Hoechst nuclear dye (Thermo Fisher Scientific), anti-MBP antibody (Abcam ab92406 or Abcam ab7349), and anti-GFAP antibody (Cell Signaling #12389) followed by goat secondary antibodies (Thermo Scientific). Images were acquired with the Arrayscan XTI instrument (Thermo Scientific) using a 10× objective, a 386/23 filter for detection of Hoechst dye, a 485/20 filter to detect MBP/Alexafluor-488 and a 549/18 filter to detect GFAP/Alexafluor-546 fluorescence. To reduce well-to-well variability, 25 images were taken covering approximately 80% of the well surface area. Images were analyzed using the HCS Studio software (Thermo Scientific). Total cell count was calculated based on the number of Hoechst⁺ nuclei. To quantify the percentage of total cells positive for either MBP or GFAP, a ring was expanded out from the nuclear mask (Hoechst dye) to include the cell body (GFAP⁺ cells). For MBP+ cells the ring was extended beyond the cell body to include OLs processes, ensuring that only mature OLs would be included in the analysis. Using the HCS Studio software the percentage of MBP⁺ and GFAP⁺ cells was calculated based on the number of MBP⁺ and GFAP⁺ cells per total number of cells. A cell was determined as positive by the software if the fluorescence intensity measured within the ring was above the threshold set for fluorescence intensity produced in secondary antibody only controls.

### Immunohistochemistry

Mice were transcardially perfused with 4% PFA under deep avertin or ketamine/xylazine anesthesia. Tissue was removed, post-fixed overnight in 4% PFA, cryoprotected in 30% sucrose/PBS, frozen in Neg-50 media (Thermo Scientific Scientific), cryosectioned into 10-12 μm sections, and placed on Tissue Tack microscope slides (Polysciences, Inc). Sections were permeabilized in 0.1-0.3% Triton X-100, blocked with 5% BSA or 5% normal donkey serum, and incubated with primary antibodies overnight at 4° C. and then fluorescent secondary antibodies for 1-2 h at room temperature. Slides were coverslipped with Prolong Gold or SlowFade Gold antifading agent with DAPI (Thermo Fisher Scientific).

The following primary antibodies were used: fibrinogen (mouse IHC: 1:1000, rabbit polyclonal, gift from J. Degen, Cincinnati); GFAP (1:200, rat monoclonal, #13-0300, Thermo Fisher Scientific); GST-pi (1:200, rabbit polyclonal, #312, MBL International), ID2 (1:2000, rabbit monoclonal, #M213, CalBioreagents); MBP (1:500, #ab7349, Abcam), OLIG-2 (1:200, rabbit polyclonal, #ab9610, EMD Millipore), PDGFRβ (1:100, goat polyclonal, #AF1042, R&D Systems), PLVAP (1:100, rat monoclonal, #553849, BD Pharmingen), VCAM-1 (1:50, rat monoclonal, #550547, BD Pharmingen).

Images were acquired with an Axioplan II epifluorescence microscope (Carl Zeiss) equipped with dry Plan-Neofluar objectives (10×0.3 NA, 20×0.5 NA, or 40×0.75 NA), an Axiocam HRc CCD camera, and the Axiovision image analysis software; the BIOREVO BZ-9000 inverted fluorescence microscope (Keyence) equipped with a Nikon CFT 60 Series infinite optical system and Keyence imaging software; or Olympus Fluoview confocal microscope equipped with 20× NA1.0 objective. All images were processed and analyzed in ImageJ. Depending on the staining, quantification was performed on thresholded, binary images or counting of cells by researchers blind to the mouse treatment group.

### Immunoblots

Cells or tissue were lysed in RIPA lysis buffer (Thermo Fisher Scientific) supplemented with protease/phosphatase inhibitor cocktails (Calbiochem) and lysates were cleared by centrifuging at 13,000×g for 15 minutes at 4° C. Equal amounts of protein were loaded in 4/o-12% bis-tris gels (Thermo Fisher Scientific) and analyzed by western blotting. Bands were visualized with HRP-conjugated secondary antibodies (Cell Signaling Technology). Densitometry was performed using ImageJ Software (NIH) with values for each band normalized to GAPDH loading controls from the same membrane. Primary antibodies were: Id2 (1:1000, rabbit monoclonal, #M213, CalBioreagents); phospho-Smad1/5 (1:1000, rabbit monoclonal, #9516, Cell Signaling Technology); GAPDH (1:1000, rabbit monoclonal, #2118, Cell Signaling Technology)

### Statistical Analyses

Statistical analyses were performed with GraphPad Prism (Version 8). Data are presented as mean t s.e.m. No statistical methods were used to predetermine sample size, but sample sizes are similar to those reported previously. Statistical significance was determined with two-sided unpaired student's t-test, or two-sided Mann-Whitney test, or a one-way or two-way analysis of variance (ANOVA) followed by Dunnett's or Tukey's post-test for multiple comparisons as indicated in the figure legends. P value ≤0.05 was considered significant. Mice with similar EAE scores (≤0.5 score difference) were randomly assigned to experimental groups and each cage had animals from each treatment group to minimize confounders. The EAE clinical scoring, histopathological analysis, and quantification were done in a blinded manner. To compare clinical scores for EAE, statistical significance of the changes in the mean clinical score for each day of the EAE experiment was estimated using permutation tests.²³ The corresponding P values were estimated using 1000 permutations. In each permutation, mice were randomly permuted. In the NOD-EAE model, means of maximum scores from the last 20 days of treatment were compared between each group with a Welch's two-sample t-test.

### BIBLIOGRAPHY

1. Zhu X, Hill R A, Dietrich D, Komitova M, Suzuki R, Nishiyama A. Age-dependent fate and lineage restriction of single NG2 cells. Development. 2011; 138(4):745-53.
2. Madisen L, Zwingman T A, Sunkin S M, et al. A robust and high-throughput Cre reporting and characterization system for the whole mouse brain. Nat Neurosci. 2010; 13(1):133-40.
3. Jung S, Aliberti J, Graemmel P, et al. Analysis of fractalkine receptor CX(3)CR1 function by targeted deletion and green fluorescent protein reporter gene insertion. Mol Cell Biol. 2000; 20(11):4106-14.
4. Mayo L, Trauger S A, Blain M, et al. Regulation of astrocyte activation by glycolipids drives chronic CNS inflammation. Nat Med. 2014; 20(10):1147-56.
5. Mendiola A S, Ryu J K, Bardehle S, et al. Transcriptional profiling and therapeutic targeting of oxidative stress in neuroinflammation. Nat Immunol. 2020; 21(5):513-524.
6. Wu Y E, Pan L, Zuo Y, Li X, Hong W. Detecting activated cell populations using single-cell RNA-seq. Neuron. 2017; 96(2):313-329 e6.
7. Robinson M D, McCarthy D J, Smyth G K. edgeR: a Bioconductor package for differential expression analysis of digital gene expression data. Bioinformatics. 2010; 26(1):139-40.
8. Zhou Y, Zhou B, Pache L, et al. Metascape provides a biologist-oriented resource for the analysis of systems-level datasets. Nat Commun. 2019; 10(1):1523.
9. Subramanian A, Tamayo P. Mootha V K, et al. Gene set enrichment analysis: a knowledge-based approach for interpreting genome-wide expression profiles. Proc Natl Acad Sci USA. 2005; 102(43):15545-50.
10. Mootha V K, Lindgren C M, Eriksson K F, et al. PGC-1alpha-responsive genes involved in oxidative phosphorylation are coordinately downregulated in human diabetes. Nat Genet. 2003; 34(3):267-73.
11. Shannon P, Markiel A, Ozier O, et al. Cytoscape: a software environment for integrated models of biomolecular interaction networks. Genome Res. 2003; 13(11):2498-504.
12. Davalos D, Lee J K, Smith W B, et al. Stable in vivo imaging of densely populated glia, axons and blood vessels in the mouse spinal cord using two-photon microscopy. J Neurosci Methods. 2008; 169(1):1-7.
13. Romanelli E, Sorbara C D, Nikić I, Dagkalis A, Misgeld T, Kerschensteiner M. Cellular, subcellular and functional in vivo labeling of the spinal cord using vital dyes. Nat Protoc. 2013; 8(3):481-490.
14. Wilke S A, Antonios J K, Bushong E A, et al. Deconstructing complexity: serial block-face electron microscopic analysis of the hippocampal mossy fiber synapse. J Neurosci. 2013; 33(2):507-22.
15. Walton J. Lead aspartate, an en bloc contrast stain particularly useful for ultrastructural enzymology. J Histochem Cytochem. 1979; 27(10):1337-42.
16. Bushong E A, Johnson D D, Jr., Kim K Y, et al. X-ray microscopy as an approach to increasing accuracy and efficiency of serial block-face imaging for correlated light and electron microscopy of biological specimens. Microsc Microanal. 2015; 21(1):231-8.
17. Kremer J R, Mastronarde D N, McIntosh J R. Computer visualization of three-dimensional image data using IMOD. J Struct Biol. 1996; 116(1):71-6.
18. Petersen M A, Ryu J K, Chang K J, et al. Fibrinogen activates BMP signaling in oligodendrocyte progenitor cells and inhibits remyelination after vascular damage. Neuron. 2017; 96(5):1003-1012 e7.
19. Mei F, Fancy S P J, Shen Y A, et al. Micropillar arrays as a high-throughput screening platform for therapeutics in multiple sclerosis. Nat Med. 2014; 20(8):954-960.
20. Najm F J, Madhavan M, Zaremba A, et al. Drug-based modulation of endogenous stem cells promotes functional remyelination in vivo. Nature. 2015; 522(7555):216-20.
21. Mei F, Mayoral S R, Nobuta H, et al. Identification of the kappa-opioid receptor as a therapeutic target for oligodendrocyte remyelination. J Neurosci. 2016; 36(30):7925-35.
22. Fancy S P, Harrington E P, Yuen T J, et al. Axin2 as regulatory and therapeutic target in newborn brain injury and remyelination. Nat Neurosci. 2011; 14(8):1009-16.
23. Ryu J K, Rafalski V A, Meyer-Franke A, et al. Fibrin-targeting immunotherapy protects against neuroinflammation and neurodegeneration. Nat Immunol. 2018:19(11):1212-1223.

### Results

NG2 cells cluster perivascularly at sites of fibrinogen deposition with limited remyelination in chronic neuroinflammation

NG2 cells, also referred to as OPCs, are progenitor cells in the adult CNS closely associated with the vasculature with unique potential to promote remyelination (Dimou and Gallo, 2015). To study NG2 cells and neurovascular dysfunction in neuroinflammation, NG2-CreER™:Rosa^{tdTomato/+}:Cx3cr1^{GFP/+} mice were generated. In vivo 2P imaging and transcriptomic profiling of NG2 cells and microglia during chronic experimental autoimmune encephalomyelitis (EAE) induced by the epitope of amino acids 35-55 of myelin oligodendrocyte glycoprotein (MOG) (‘MOG₃₅₋₅₅ EAE’) were performed (Supplementary FIG. 1). Extravasation of 70 kilodalton Oregon Green Dextran was used as a marker of acute BBB leakage, and fibrinogen immunohistology as a marker of chronic BBB leakage and local coagulation. At peak EAE, perivascular clusters consisted primarily of microglia, and NG2 cells were evenly distributed in the spinal cord parenchyma (FIG. 1A, Supplementary FIG. 2A). However, in chronic EAE, perivascular clusters also consisted of NG2 cells, with more than ˜80% of NG2 cell clusters located at or within 30 μm of a blood vessel (FIG. 1A, Supplementary FIG. 2B). NG2^{tdTomato+} cells in the clusters had glial-like morphology characterized by multiple branched processes in the spinal cord parenchyma, distinguishable from NG2^{tdTomato+} pericytes with elongated processes along the blood vessel wall (Supplementary FIG. 2C). VCAM1, a marker of endothelial activation (Lengfeld et al., 2017), and PLVAP, a marker of endothelial fenestrae in leaky CNS vessels (Niu et al., 2019), were increased in peak and chronic EAE white matter (Supplementary FIG. 3A, B), suggesting disruption of neurovascular homeostasis. Fibrinogen deposition is a prominent feature of neurovascular pathology in EAE, necessary for disease pathogenesis (Adams et al., 2007; Davalos et al., 2012; Ryu et al., 2018). While acute dextran leakage was highest at peak EAE, fibrinogen deposition increased over time and was highest during chronic EAE (FIG. 1B), suggesting persistent fibrinogen deposition even when active BBB disruption declined. In chronic EAE, NG2 clusters aggregated perivascularly only at sites of fibrinogen deposition (FIG. 1C, Supplementary FIG. 4A), and often co-localized with microglial clusters (FIG. 1A, Supplementary FIG. 2A). These results suggest dynamic glial remodeling of the neurovascular interface at sites of fibrinogen deposition during neuroinflammation.

To assess myelin within perivascular NG2 clusters using in vivo 2P imaging, MitoTracker Deep Red, a mitochondrial dye that also labels myelin when used at higher concentrations (Romanelli et al., 2013), was applied. Significant myelin disruption, characterized by blebbing of myelin sheaths, was present near NG2 clusters, whereas normal-appearing myelin sheaths appeared at sites without clusters (FIG. 1D, Supplementary FIG. 4B). To study myelin ultrastructure, a co-registration technique was developed to correlate 2P-imaged volume with three-dimensional serial block face electron microscopy (SBEM) using microcomputed tomography (FIG. 1E). Using this technique, SBEM images were collected at the exact same areas of perivascular NG2 clusters in EAE mice imaged by in vivo 2P microscopy. Inflamed veins with endothelial activation, attachment of leukocytes at the endothelial surface, perivascular astrogliosis, and inflammation, in part with debris-containing macrophages were observed (FIG. 1Fi, Gi, Supplementary FIG. 3C). In the parenchymal lesions we found two distinct patterns: the first was characterized by cell infiltration of elongated cells with low cell density, some of which contained osmiophilic degradation products. In these areas, axons were predominantly demyelinated, and remyelination was sparse (FIG. 1Fii, Gi). In other areas, there were more dense clusters of small cells with small rims of perinuclear cytoplasm containing some mitochondria, but few other organelles, which were reminiscent of NG2 cells (FIG. 1Gii). Remyelinated axons were closely adjacent to these cell clusters, while in areas distant from the clusters, axons were demyelinated (FIG. 1Fiii, Gii). Away from perivascular NG2 cells, normal-appearing perivascular CNS tissue, astrocytic glia limitans, and axons with normal myelin thickness were observed (FIG. 1Fiv). These results suggest that perivascular NG2 clusters are associated with inflammation, gliosis, frank demyelination and limited remyelination. Transcriptomic profiling of NG2 cells in EAE reveals suppression of vascular homeostasis and anticoagulation pathways

To study the transcriptomic changes in NG2 cells in chronic EAE, RNA-seq was performed on NG2^{tdTomato+} cells collected from the spinal cords of MOG₃₅₋₅₅ EAE mice or healthy controls (Supplementary FIG. 3A). A total of 1,241 differentially expressed genes (DEGs) (FDR<0.05; ±1 log₂ fold change) were identified in the setting of chronic EAE compared to control, of which 738 were downregulated (60%) and 503 upregulated (40%) (FIG. 2A). Unsupervised gene clustering analysis identified 9 distinct gene clusters (FIG. 2B). Gene ontology (GO) analysis revealed that chronic EAE activated inflammatory and antigen-presentation genes in clusters 1-4, including the GO pathway terms “Positive regulation of acute inflammatory response,” “Positive regulation of T cell mediated cytotoxicity,” “Antigen processing and presentation,” and “Cellular response to interferon-beta” (FIG. 2B, Supplementary Table 1). Canonical antigen presentation genes, such as Cd74, H2-dma, and B2m, were significantly upregulated in EAE (FIG. 2B), consistent with reports suggesting immune-like functions of OL lineage cells in disease (Falcao et al., 2018; Kirby et al., 2019). Interestingly, GO analysis of downregulated gene clusters 5-9 revealed pathways related to vascular and BBB homeostasis, such as “Angiogenesis,” “Regulation of Wnt signaling pathway,” “Vasculogenesis,” “Blood vessel development,” and “Cell junction organization” (FIG. 2B). In accordance, gene networks involved in blood vessel maintenance, wound healing and coagulation, and tight junctions were globally repressed in EAE (FIG. 2C). Gene set enrichment analysis (GSEA) of DEGs identified the top two downregulated gene sets as “Regulation of cell junction assembly” (normalized enrichment score (NES) 1.7, p<0.01) and “Negative regulation of coagulation” (NES 1.7, p<0.01) (FIG. 2D). Expression of tissue factor pathway inhibitor (Tfpi), a primary inhibitor of blood coagulation and fibrin formation (Wood et al., 2014), was significantly reduced in NG2 cells in EAE. As the NG2^{tdTomato+} population includes OPC and pericyte lineages (Supplementary FIG. 6), we isolated PDGFRα⁺ OPCs and PDGFRβ⁺ pericytes from the spinal cords of MOG₃₅₋₅₅-EAE mice or healthy controls (Supplementary FIG. 3B) and labeled cell surface major histocompatibility complex class II (MHCII) and TFPI to assess the antigen presentation and anticoagulation pathways, respectively. Consistent with our bulk-RNAseq and prior studies (Kirby et al., 2019), MHCII was increased in PDGFRα⁺ OPCs in EAE (Supplementary FIG. 3C). TFPI was expressed by OPCs but not pericytes in healthy controls and was significantly repressed in EAE (FIG. 2E, F). Overall, these results identify dysregulation of antigen presentation, coagulation, and vascular homeostasis pathways in OPCs in chronic neuroinflammation.

Promyelinating compounds do not overcome fibrinogen extrinsic inhibition of OPC differentiation

OPCs can differentiate to myelinating OLs or astrocyte-like cells in response to extrinsic signals found in multiple sclerosis lesions like fibrinogen or BMPs (Mabie et al., 1997; Petersen et al., 2017. Hackett et al., 2018). We developed the OPC-X-screen, a medium-throughput, high-content imaging assay to identify compounds that in the presence of extrinsic inhibitors promote OPC differentiation to mature MBP⁺ OLs and decrease the OPC fate-switch to GFAP⁺ astrocytes (FIG. 3A). In the OPC-X assay, fibrinogen decreased MBP⁺ mature OLs and increased GFAP⁺ astrocyte-like cells by ˜60% as compared to controls (FIG. 3B-D). Seven compounds—benztropine, clemastine, quetiapine, miconazole, clobetasol, (±)U-50488, and XAV-939—have been previously identified to promote intrinsic pathways of OPC differentiation (Fancy et al., 2011; Mei et al., 2014; Najm et al., 2015; Mei et al., 2016). However, these promyelinating compounds did not overcome extrinsic inhibition of OPC differentiation by fibrinogen (FIG. 3B-D). In contrast, the BMP receptor inhibitor DMH1 (Hao et al., 2010) rescued the inhibitory effects of fibrinogen and restored OPC differentiation to mature OLs to control levels (FIG. 3B-D). Cell-fate switch of OPCs to GFAP⁺ cells by fibrinogen was also abolished by DMH1 (FIG. 3D). Clemastine, a muscarinic receptor antagonist, promotes the remyelinating potential of OPCs and is currently in clinical trials for multiple sclerosis (Mei et al., 2014; Green et al., 2017). While clemastine increased the number of MBP⁺ cells in control conditions as expected, it did not enhance OPC differentiation to mature OLs in the presence of fibrinogen (Supplementary FIG. 4). Clemastine did not block fibrinogen-induced phosphorylation of the BMP signal transducers SMAD1/5 or expression of the BMP target protein ID2 (FIG. 3E). In contrast, DMH1 blocked fibrinogen induced SMAD1/5 phosphorylation and ID2 expression (FIG. 3E). Thus, previously identified compounds promoting OPC differentiation may not overcome extrinsic inhibition signaling pathways at sites of vascular damage.

Therapeutic effects of type 1 BMP receptor inhibition in neuroinflammation BMP expression and downstream receptor signaling is increased in human multiple sclerosis lesions (Costa et al., 2019; Harnisch et al., 2019). The BMP target protein ID2 is also increased in lesions with extensive fibrinogen deposition (Petersen et al., 2017). The finding that DMH1 effectively blocked fibrinogen-induced BMP receptor activation and restored OPC differentiation in vitro (FIG. 3) suggested that targeting BMP signaling may promote repair in neuroinflammation. However, DMH1 is not water-soluble, which limits its use in vivo. Therefore, we tested LDN-212854, a water-soluble activin A receptor type I (ACVR1)-biased type I BMP receptor inhibitor with a molecular structure similar to DMH1 (Mohedas et al., 2013), in the OPC-X-Screen. LDN-212854 restored mature OL differentiation and blocked the formation of GFAP+ astrocytes from fibrinogen-treated OPCs in a dose-dependent manner (FIG. 3F,G).

To determine the therapeutic potential of LDN-212854, we selected two models of EAE: chronic MOG₃₅₋₅₅ EAE induced in NG2-CreER™:Rosa^{tdTomato/+} mice and progressive EAE induced in non-obese diabetic (NOD) mice by the epitope of amino acids 35-55 of MOG (‘NOD-MOG₃₅₋₅₅ EAE’) (Mayo et al., 2014). Therapeutic administration of LDN-212854 significantly improved clinical scores (FIG. 4A-D) and reduced fibrinogen deposition and demyelination in both models (FIG. 4A-D). LDN-212854 also markedly reduced perivascular NG2 clusters and myelin damage in MOG₃₅₋₅₅ EAE, as revealed by in vivo 2P imaging (FIG. 4E, F). Moreover, LDN-212854 decreased ID2 expression in NG2 cells in the EAE white matter (FIG. 4G), consistent with inhibition of BMP signaling in the NG2 cell lineage.

Since a key mechanism of fibrinogen and BMP receptor signaling is cell fate switch of OPCs to astrocytes (Mabie et al., 1997; Petersen et al., 2017), we tested whether LDN-212854 promoted OPC differentiation to myelinating cells in MOG35-55 EAE. To trace the cell fate of OPCs in vivo, we induced EAE in the NG2-CreER™:Rosa^{tdTomato/+} mice, allowing tamoxifen-induced expression of tdTomato in NG2⁺ OPCs and their progeny (Petersen et al., 2017; Hackett et al., 2018). Glutathione s-transferase-pi (GST-pi) labeled mature OLs and GFAP labeled astrocytes derived from genetically-labeled tdTomato⁺ NG2⁺ OPCs. Therapeutic administration of LDN-212854 increased the proportion NG2^{tdTomato+} OPCs that differentiated into GST-pi⁺ mature OLs compared to controls, and abolished formation of OPC-derived GFAP⁺ astrocytes in NG2-CreER™:Rosa^{tdTomato+} MOG₃₅₋₅₅ EAE mice (FIG. 4H). Collectively, these results suggest that Type I BMP receptor inhibition restores the cell fate of OPCs to mature OLs with therapeutic potential in neuroinflammatory disease with fibrinogen deposition and active BMP signaling.

Discussion

The data provided herein reveals dynamic cellular remodeling of the neurovascular niche at sites of BBB dysfunction in neuroinflammation and identifies a druggable pathway to promote myelin repair. Perhaps in neuroinflammation, perivascular NG2⁺ OPC clusters contribute to a procoagulant environment leading to excessive fibrinogen deposition, activation of BMP receptor signaling in OPCs, and extrinsic inhibition of remyelination at sites of vascular damage. This model is consistent with chronically demyelinated multiple sclerosis lesions, in which perivascular OPC clusters are localized in the active lesion borders with fibrinogen deposition, impaired fibrinolysis, BMP pathway activation, and gliosis (Petersen et al., 2017; Yates et al., 2017; Lee et al., 2018; Niu et al., 2019). Through the OPC-X-screen, we discovered that the therapeutic potential of many promyelinating drugs may be limited at sites of vascular damage and fibrinogen deposition, highlighting the unmet clinical need for therapeutic strategies to overcome extrinsic inhibition in diseases with chronic demyelination. Provided herein is the concept that inhibiting BMP pathway activation can promote myelin repair by overcoming abortive OPC differentiation at sites of neurovascular dysfunction. Thus, BMP inhibitors can expand the toolbox of promyelinating drugs and provide additional therapeutic options for patients with BBB disruption and white matter pathology.

Using in vivo 2P imaging, we found a striking transition of the perivascular glial cell composition associated with microglia and demyelination at the peak of disease, followed by the formation of perivascular NG2 clusters with limited remyelination in chronic neuroinflammation. NG2 cell clustering at sites of fibrinogen deposition suggests that OPC migration or adhesion may be altered at sites of vascular damage or that OPCs themselves may contribute to BBB disruption or local coagulation. This study suggests previously unknown functions of OPCs in the expression of genes regulating coagulation. TFPI, a potent inhibitor of coagulation factor X and tissue factor-mediated coagulation (Wood et al., 2014), was expressed in OPCs and repressed by chronic neuroinflammation. Interestingly, multiple sclerosis patients have alterations in hemostasis biomarkers including TFPI (Ziliotto et al., 2019), suggesting an imbalance in anti- and procoagulant pathways in neuroinflammatory disease. Prooxidant microglia may also contribute to the procoagulant milieu in the lesion microenvironment through expression of coagulation proteins such as coagulation factor X (Mendiola et al., 2020). Thus, transcriptional changes at the neurovascular interface may establish a local procoagulant environment that contributes to the excessive or persistent deposition of fibrin observed in many neurological diseases (Petersen et al., 2018). Therapeutic strategies to target the NG2 cell-vascular-fibrinogen axis or downstream fibrinogen signaling can provide a therapeutic avenue to overcome extrinsic inhibition in the neuroinflammatory lesion environment.

The study suggests that promyelinating drugs differentially suppress signaling pathways activated by extrinsic inhibitors in the lesion environment. Indeed, clemastine did not inhibit SMAD1/5 phosphorylation, a key pathway downstream of BMP receptor activation, or rescue OPC cell fate switch to astrocytes. Fibrinogen, in addition to activating BMP receptor signaling in OPCs, stimulates CSPG production from astrocytes and is a carrier for transforming growth factor-beta (TGF-β) (Schachtrup et al., 2010). CSPGs inhibit remyelination in part through activation of the protein tyrosine phosphatase sigma receptor in OPCs (Pendleton et al., 2013). Age-related loss of OPC function may occur in response to TGF-β signaling or increased stiffness in the OPC niche, with subsequent signaling through the mechanoresponsive ion channel Piezol (Baror et al., 2019; Segel et al., 2019). Therefore, assays that better recapitulate the inhibitory lesion environment and downstream signaling are needed to improve selection of drugs that can increase remyelination in inflammatory lesions with gliosis, vascular damage and BBB disruption. Furthermore, the choice of promyelinating drug in the clinic may need to take into account its efficacy within the extrinsic inhibitory milieu in patients with demyelinating neurological diseases. Targeting multiple inhibitory pathways with combinations of drugs may have additive or synergistic effects on remyelination and could provide an avenue to maximize the therapeutic benefit of promyelinating compounds in an inhibitory lesion environment.

Therapeutic fibrinogen depletion by anticoagulants can suppress neuroinflammation and promote myelin regeneration (Akassoglou et al., 2002; Petersen et al., 2017), but hemorrhagic complications may limit the clinical utility of this approach. The instant study identifies LDN-212854, an ACVR1-biased BMP receptor inhibitor, as a potential therapeutic agent for chronic neuroinflammation. Activation of fibrinogen and BMP signaling in the injured perivascular niche directs OPC cell fate towards astrocytes rather than remyelinating OLs (Petersen et al., 2017; Baror et al., 2019), which may contribute to pathologic gliosis at sites of vascular damage. LDN-212854 increased myelinating OLs and eliminated OPC differentiation to astrocytes. LDN-212854 was well-tolerated at the doses used in the study, but human toxicity data is limited. Clinical use of ACVR1-selective BMP inhibitors has gained recent attention for the treatment of fibrodysplasia ossificans progressive, a rare disorder with overactive BMP signaling resulting in heterotopic ossification and myelin abnormalities (Kan et al., 2012). LDN-212854 and other safe ACVR1-selective inhibitors may be a therapeutic option for neurological diseases with BBB disruption and myelin abnormalities including multiple sclerosis, Alzheimer disease, neonatal brain injury, and traumatic brain injury.

### BIBLIOGRAPHY

Adams R A, Bauer J, Flick M J, Sikorski S L, Nuriel T, Lassmann H, et al. The fibrin-derived gamma377-395 peptide inhibits microglia activation and suppresses relapsing paralysis in central nervous system autoimmune disease. J Exp Med 2007; 204(3): 571-82.
Akassoglou K, Yu W-M, Akpinar P, Strickland S. Fibrin inhibits peripheral nerve regeneration by arresting Schwann cell differentiation. Neuron 2002; 33: 861-75.
Back S A, Gan X, Li Y, Rosenberg P A, Volpe J J. Maturation-dependent vulnerability of oligodendrocytes to oxidative stress-induced death caused by glutathione depletion. J Neurosci 1998; 18(16): 6241-53.
Baror R, Neumann B, Segel M, Chalut K J, Fancy S P J, Schafer D P, et al. Transforming growth factor-beta renders ageing microglia inhibitory to oligodendrocyte generation by CNS progenitors. Glia 2019; 67(7): 1374-84.
Costa C, Eixarch H, Martinez-Saez E, Calvo-Barreiro L, Calucho M, Castro Z, et al. Expression of bone morphogenetic proteins in multiple sclerosis lesions. Am J Pathol 2019; 189(3): 665-76.
Davalos D, Ryu J K, Merlini M, Baeten K M, Le Moan N, Petersen M A, et al. Fibrinogen-induced perivascular microglial clustering is required for the development of axonal damage in neuroinflammation. Nat Commun 2012; 3: 1227.
Deshmukh V A, Tardif V, Lyssiotis C A, Green C C, Kerman B, Kim H J, et al. A regenerative approach to the treatment of multiple sclerosis. Nature 2013; 502(7471): 327-32.
Dimou L, Gallo V. NG2-glia and their functions in the central nervous system. Glia 2015; 63(8): 1429-51.
Falcao A M, van Bruggen D, Marques S, Meijer M, Jakel S, Agirre E, et al. Disease-specific oligodendrocyte lineage cells arise in multiple sclerosis. Nat Med 2018; 24(12): 1837-44.
Fancy S P, Harrington E P, Yuen T J, Silbereis J C, Zhao C, Baranzini S E, et al. Axin2 as regulatory and therapeutic target in newborn brain injury and remyelination. Nat Neurosci 2011; 14(8): 1009-16.
Forbes T A, Gallo V. All wrapped up: environmental effects on myelination. Trends Neurosci 2017; 40(9): 572-87.
Franklin R J M, Ffrench-Constant C. Regenerating CNS myelin—from mechanisms to experimental medicines. Nat Rev Neurosci 2017, 18(12): 753-69.
Green A J, Gelfand J M, Cree B A, Bevan C, Boscardin W J, Mei F, et al. Clemastine fumarate as a remyelinating therapy for multiple sclerosis (ReBUILD): a randomised, controlled, double-blind, crossover trial. Lancet 2017; 390(10111): 2481-9.
Hackett A R, Yahn S L, Lyapichev K, Dajnoki A, Lee D H, Rodriguez M, et al. Injury type-dependent differentiation of NG2 glia into heterogeneous astrocytes. Exp Neurol 2018; 308: 72-9.
Hao J, Ho J N, Lewis J A, Karim K A, Daniels R N, Gentry P R, et al. In vivo structure-activity relationship study of dorsomorphin analogues identifies selective VEGF and BMP inhibitors. ACS Chem Biol 2010; 5(2): 245-53.
Harnisch K, Teuber-Hanselmann S, Macha N, Mairinger F, Fritsche L, Soub D, et al. Myelination in multiple sclerosis lesions is associated with regulation of bone morphogenetic protein 4 and its antagonist noggin. Int J Mol Sci 2019; 20(1).
Kan L, Kitterman J A, Procissi D, Chakkalakal S, Peng C Y, McGuire T L, et al. CNS demyelination in fibrodysplasia ossificans progressiva. J Neurol 2012; 259(12): 2644-55.
Keough M B, Rogers J A, Zhang P, Jensen S K, Stephenson E L, Chen T, et al. An inhibitor of chondroitin sulfate proteoglycan synthesis promotes central nervous system remyelination. Nat Commun 2016; 7: 11312.
Kirby L, Jin J, Cardona J G, Smith M D, Martin K A, Wang J, et al. Oligodendrocyte precursor cells present antigen and are cytotoxic targets in inflammatory demyelination. Nat Commun 2019; 10(1): 3887.
Lee N J, Ha S K, Sati P, Absinta M, Luciano N J, Lefeuvre J A, et al. Spatiotemporal distribution of fibrinogen in marmoset and human inflammatory demyelination. Brain 2018; 141(6): 1637-49.
Lengfeld J E, Lutz S E, Smith J R, Diaconu C, Scott C, Kofman S B, et al. Endothelial Wnt/beta-catenin signaling reduces immune cell infiltration in multiple sclerosis. Proc Natl Acad Sci USA 2017; 114(7): E1168-E77.
Mabie P C, Mehler M F, Marmur R, Papavasiliou A, Song Q, Kessler J A. Bone morphogenetic proteins induce astroglial differentiation of oligodendroglial-astroglial progenitor cells. J Neurosci 1997; 17(11): 4112-20.
Magliozzi R, Hametner S, Facchiano F, Marastoni D, Rossi S, Castellaro M, et al. Iron homeostasis, complement, and coagulation cascade as CSF signature of cortical lesions in early multiple sclerosis. Ann Clin Transl Neurol 2019; 6(11): 2150-63.
Mayo L, Trauger S A, Blain M, Nadeau M, Patel B, Alvarez J I, et al. Regulation of astrocyte activation by glycolipids drives chronic CNS inflammation. Nat Med 2014; 20(10): 1147-56.
Mei F, Fancy S P J, Shen Y A, Niu J, Zhao C, Presley B, et al. Micropillar arrays as a high-throughput screening platform for therapeutics in multiple sclerosis. Nat Med 2014; 20(8): 954-60.
Mei F, Mayoral S R, Nobuta H, Wang F, Desponts C, Lorrain D S, et al. Identification of the Kappa-Opioid Receptor as a Therapeutic Target for Oligodendrocyte Remyelination. J Neurosci 2016; 36(30): 7925-35.
Mendiola A S, Ryu J K, Bardehle S, Meyer-Franke A, Ang K K, Wilson C, et al. Transcriptional profiling and therapeutic targeting of oxidative stress in neuroinflammation. Nat Immunol 2020; 21(5): 513-24.
Miron V E, Boyd A, Zhao J W, Yuen T J, Ruckh J M, Shadrach J L, et al. M2 microglia and macrophages drive oligodendrocyte differentiation during CNS remyelination. Nat Neurosci 2013; 16(9): 1211-8.
Mohedas A H, Xing X, Armstrong K A, Bullock A N, Cuny G D, Yu P B. Development of an ALK2-biased BMP type I receptor kinase inhibitor. ACS Chem Biol 2013; 8(6): 1291-302.
Najm F J, Madhavan M, Zaremba A, Shick E, Karl R T, Factor D C, et al. Drug-based modulation of endogenous stem cells promotes functional remyelination in vivo. Nature 2015; 522(7555): 216-20.
Neumann B, Baror R, Zhao C, Segel M, Dietmann S, Rawji K S, et al. Metformin restores CNS remyelination capacity by rejuvenating aged stem cells. Cell Stem Cell 2019; 25(4): 473-85 e8.
Niu J, Tsai H H, Hoi K K, Huang N, Yu G, Kim K, et al. Aberrant oligodendroglial-vascular interactions disrupt the blood-brain barrier, triggering CNS inflammation. Nat Neurosci 2019; 22(5): 709-18.
Pendleton J C, Shamblott M J, Gary D S, Belegu V, Hurtado A, Malone M L, et al. Chondroitin sulfate proteoglycans inhibit oligodendrocyte myelination through PTPsigma. Exp Neurol 2013; 247: 113-21.
Petersen M A, Ryu J K, Akassoglou K. Fibrinogen in neurological diseases: mechanisms, imaging and therapeutics. Nat Rev Neurosci 2018; 19(5): 283-301.
Petersen M A, Ryu J K, Chang K J, Etxeberria A, Bardehle S, Mendiola A S, et al. Fibrinogen activates BMP signaling in oligodendrocyte progenitor cells and inhibits remyelination after vascular damage. Neuron 2017; 96(5): 1003-12 e7.
Pous L, Deshpande S S, Nath S, Mezey S, Malik S C, Schildge S, et al. Fibrinogen induces neural stem cell differentiation into astrocytes in the subventricular zone via BMP signaling. Nat Commun 2020; 11(1): 630.
Reich D S, Lucchinetti C F, Calabresi P A. Multiple Sclerosis. N Engl J Med 2018; 378(2): 169-80.
Romanelli E, Sorbara C D, Nikic I, Dagkalis A, Misgeld T, Kerschensteiner M. Cellular, subcellular and functional in vivo labeling of the spinal cord using vital dyes. Nat Protoc 2013; 8(3): 481-90.
Ryu J K, Petersen M A, Murray S G, Baeten K M, Meyer-Franke A, Chan J P, et al. Blood coagulation protein fibrinogen promotes autoimmunity and demyelination via chemokine release and antigen presentation. Nat Commun 2015; 6: 8164.
Ryu J K, Rafalski V A, Meyer-Franke A, Adams R A, Poda S B, Rios Coronado P E, et al. Fibrintargeting immunotherapy protects against neuroinflammation and neurodegeneration. Nat Immunol 2018; 19(11): 1212-23.
Schachtrup C, Ryu J K, Helmrick M J, Vagena E, Galanakis D K, Degen J L, et al. Fibrinogen triggers astrocyte scar formation by promoting the availability of active TGF-beta after vascular damage. J Neurosci 2010; 30(17): 5843-54.
Segel M, Neumann B, Hill M F E, Weber I P, Viscomi C, Zhao C, et al. Niche stiffness underlies the ageing of central nervous system progenitor cells. Nature 2019; 573(7772): 130-4.
Starost L, Lindner M, Herold M, Xu Y K T, Drexler H C A, Hess K, et al. Extrinsic immune cell derived, but not intrinsic oligodendroglial factors contribute to oligodendroglial differentiation block in multiple sclerosis. Acta Neuropathol 2020; 140(5): 715-36.
Vos C M, Geurts J J, Montagne L, van Haastert E S, Bo L, van der Valk P, et al. Blood-brain barrier alterations in both focal and diffuse abnormalities on postmortem MRI in multiple sclerosis. Neurobiol Dis 2005; 20(3); 953-60.
Wood J P, Ellery P E, Maroney S A, Mast A E. Biology of tissue factor pathway inhibitor. Blood 2014; 123(19): 2934-43.
Yates R L, Esiri M M, Palace J, Jacobs B, Perera R, DeLuca G C. Fibrin(ogen) and neurodegeneration in the progressive multiple sclerosis cortex. Ann Neurol 2017; 82(2): 259-70.
Ziliotto N, Bernardi F, Jakimovski D, Zivadinov R Coagulation pathways in neurological diseases: multiple sclerosis. Front Neurol 2019; 10: 409.

All publications, nucleotide and amino acid sequence identified by their accession nos., patents and patent applications are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

The specific methods and compositions described herein are representative of embodiments and are exemplary and not intended as limitations on the scope of the invention. Other objects, aspects, and embodiments will occur to those skilled in the art upon consideration of this specification and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. The methods and processes illustratively described herein suitably may be practiced in differing orders of steps, and the methods and processes are not necessarily restricted to the orders of steps indicated herein or in the claims.

Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by embodiments and optional features, modification and variation of the concepts herein disclosed may be resorted to by those skilled in the art, and that such modifications and variations are considered to be within the scope of this invention as defined by the appended claims and statements of the invention.

1. A method to treat or prevent neurodegeneration in a mammal comprising administering to the mammal in need thereof an effective amount of an inhibitor of at least one bone morphogenetic protein (BMP) receptor.

2. A method to treat or prevent neurodegeneration in a mammal comprising administering to the mammal in need thereof an effective amount of an inhibitor of ACVR1 (Alk2) or an agent that modulate the ligand for ACVR1 (activin).

3. A method to promote remyelination in neurological diseases or disorders in a mammal, comprising administering to the mammal in need thereof an effective amount of an inhibitor of ACVR1 (Alk2) or an agent that modulate the ligand for ACVR1 (activin).

4. A method to prevent or ameliorate demyelination in a mammal comprising administering to the mammal in need thereof an effective amount of an inhibitor of ACVR1 (Alk2) or an agent that modulate the ligand for ACVR1 (activin) or an agent that modulate the ligand for ACVR1 (activin).

5. A method to enhance myelination and/or re-myelination in a mammalian subject, such as a human subject, by administering to the mammal in need thereof an effective amount of an inhibitor of ACVR1 (Alk2) or an agent that modulate the ligand for ACVR1 (activin).

6. A method to decrease differentiation of progenitors to astrocytes in a mammalian subject, such as a human subject, by administering to the mammal in need thereof an effective amount of an inhibitor of ACVR1 (Alk2) or an agent that modulate the ligand for ACVR1 (activin).

7. The method of any one of claim 1, wherein the inhibitor is of ACVR1 (Alk2) is LDN-212854, dorsomorphin, DMH1, saracatinib, BCX9250, KER-047, INCB000928, BLU-782, momelotinib, LDN-193189, K02288, LDN-214117, LDN-213844, M4K2009, M4K2149 or derivatives or variants thereof.

8. The method of any one of claim 1, wherein the mammal is human.

9. The method of any one of claim 1, wherein the mammal has been diagnosed with a disease, disorder, or injury involving demyelination, dysmyelination, or neurodegeneration. In one embodiment, said disease, disorder, or injury is selected from the group consisting of multiple sclerosis (MS), progressive multifocal leukoencephalopathy (PML), encephalomyelitis (EPL), central pontine myelolysis (CPM), adrenoleukodystrophy, Alexander's disease, Pelizaeus Merzbacher disease (PMZ), Wallerian Degeneration, optic neuritis, transverse myelitis, amyotrophic lateral sclerosis (ALS), Huntington's disease, Alzheimer's disease, Parkinson's disease, spinal cord injury, traumatic brain injury, neonatal brain injury, post radiation injury, neurologic complications of chemotherapy, stroke, acute ischemic optic neuropathy, vitamin E deficiency, isolated vitamin E deficiency syndrome, AR, Bassen-Kornzweig syndrome, Marchiafava-Bignami syndrome, metachromatic leukodystrophy, trigeminal neuralgia, acute disseminated encephalitis, Guillain-Barre syndrome, Marie-Charcot-Tooth disease and Bell's palsy.

10. The method of claim 9, wherein an additional agent is administered in the treatment of Alzheimer's disease, wherein said additional agent is an acetylcholinesterase inhibitor (e.g., donepezil, galantamine, and rivastigmine) and/or NMDA receptor antagonist (e.g., memantine).

11. The method of claim 9, wherein an additional agent is administered in the treatment of ALS, wherein said additional agent is Riluzole (Rilutek), minocycline, insulin-like growth factor 1 (IGF-1), and/or methylcobalamin.

12. The method of claim 9, wherein an additional agent is administered in the treatment of Parkinson's disease, wherein said additional agent is a L-dopa, dopamine agonist (e.g., bromocriptine, pergolide, pramipexole, ropinirole, cabergoline, apomorphine, and lisuride), dopa decarboxylase inhibitor (e.g., levodopa, benserazide, and carbidopa), and/or MAO-B inhibitor (e.g., selegiline and rasagiline).

13. The method of claim 9, wherein an additional agent is administered in the treatment of demyelinating diseases, wherein said additional agent is an interferon beta la inhibitor, interferon beta lb inhibitor, glatiramer acetate, daclizumab, teriflunomide, clemestine, fingolimod, dimethyl fumarate; alemtuzumab, mitoxantrone, and/or natalizumab.

14. The method of any one of claim 1 further comprising administering an additional promyelinating agent/drug.

15. The method of 14, wherein promyelinating agent/drug is a promyelinating benztropine, clemastine, quetiapine, miconazole, clobetasol, (±)U-50488, and XAV-939.

16. The method of any one of claim 1, wherein the agent that modulates the ligand for ACVR1 (activin) is an antibody.

17. The method of claim 16, wherein the antibody is REGN2477.
